(19) United States
(12) Patent Application Publication
(10) Pub. No.: US 2022/0265298 A1
(43) Pub. Date: Aug. 25, 2022

(54) DEVICES, SYSTEMS, AND METHODS FOR CATARACT PROCEDURES

(71) Applicant: LytArc, Inc., La Jolla, CA (US)

(72) Inventors: David Scott MICHELSON, La Jolla, CA (US); Herbert Tsvi GOLDENBERG, La Jolla, CA (US); Hanan NISAN, La Jolla, CA (US)

(21) Appl. No.: 17/740,720

(22) Filed: May 10, 2022

Related U.S. Application Data
(63) Continuation of application No. PCT/US2020/059962, filed on Nov. 11, 2020.
(60) Provisional application No. 62/934,349, filed on Nov. 12, 2019.

(51) Int. Cl. A61B 17/32

(52) U.S. Cl. CPC A61B 17/32002; A61B 2018/1422

(57) ABSTRACT

A cutting device comprising a curved cutting head and an oscillator for use in medical procedures, such as cataract surgery. The cutting head of the cutting device can be sized and shaped to improve precision and reproducibility of incisions made into biological tissue. In some embodiments, a cutting head of the cutting device can comprise a bent distal tip for making incisions into biological tissue, such as a capsule of an eye.

### CROSS-REFERENCE

This application is a Continuation of International Application No. PCT/US2020/059962, filed Nov. 11, 2020, which claims the benefit of U.S. Provisional Application No. 62/934,349, filed Nov. 12, 2019, which are hereby incorporated by reference in their entirety herein.

### SUMMARY

The present disclosure generally relates to systems, devices, and methods useful in treating an eye condition in a patient and more particularly relates to devices, systems and methods useful for treating cataracts in a patient. Disclosed herein are devices, systems, and/or methods for accessing, fragmenting, emulsifying, and/or removing all or a portion of an eye lens (e.g., during a cataract removal procedure).

Instruments used in current cataract surgery techniques may be inefficient or prohibitively expensive in many common situations. For example, ultrasound and femtolaser-based techniques may not be effective in the fragmentation or emulsification of lenses in subjects with hard cataracts or unstable (e.g., “weak” or “loose”) zonules. Existing instruments used in the fragmentation of a subject's lens (e.g., such as “choppers” and “nucleus crackers”) can require excessive sawing motions and can be overly specialized, requiring a practitioner to change instruments multiple times throughout the course of accessing and fragmenting a lens during an eye procedure. In developing nations, equipment used in ultrasound- and femtolaser-based lens fragmentation and emulsification is often prohibitively expensive and logistically difficult to import or repair.

Provided herein is a device for performing a medical procedure such as cutting tissue of a subject, the device comprising: a cutting head comprising: a distal tip comprising a first distal tip edge; a proximal end; and a first portion between the distal tip and the proximal end, the first portion comprising first edge and a second edge, at least one of which is a sharp edge, wherein the first distal tip edge of the distal tip forms a first angle with the first edge of the first portion of between 0 degrees and 180 degrees; and an oscillator operatively coupled to the proximal end of the cutting head. In some embodiments, the distal tip comprises a second distal tip edge and the first distal tip edge of the distal tip forms a second angle with a second distal tip edge of the distal tip of from 90 degrees to 180 degrees, between 90 degrees and 135 degrees, or from 135 degrees to less than 180. In some embodiments, the first angle is between 0 degrees and 90 degrees, from 1 degree to 30 degrees, from 30 degrees to 45 degrees, from 45 degrees to 60 degrees, from 30 degrees to 60 degrees, or from 60 degrees to less than 90 degrees. In some embodiments, the first portion is curved. In some embodiments, the first portion has a radius of curvature of at least 1 mm, at least 2 mm, at least 3 mm, at least 4 mm, at least 5 mm, at least 6 mm, at least 7 mm, at least 8 mm at least 9 mm, at least 10 mm, more than 10 mm, from 1 mm to 10 mm, from 2 mm to 9 mm, from 2 mm to 8 mm, from 2 mm to 7 mm, from 2 mm to 6 mm, from 2 mm to 5 mm, from 2 mm to 4 mm, or from 2 mm to 3 mm. In some embodiments, the first portion has a radius of curvature of 2.75 mm. In some embodiments, the first portion is semicircular. In some embodiments, the second edge and the third edge of the first portion are both sharp edges. In some embodiments, the first distal tip edge of the distal tip is sharp. In some embodiments, the second distal tip edge of the distal tip is sharp. In some embodiments, a medial surface of the distal tip forms a second angle with a lateral surface of the distal tip of between 0 degrees and 90 degrees, from 1 degrees to 75 degrees, from 15 degrees to 85 degrees, from 20 degrees to 70 degrees, from 10 degrees to 45 degrees, from 30 degrees to 60 degrees, from 25 degrees to 65 degrees, from 15 degrees to 90 degrees, or from 15 degrees to 75 degrees. In some embodiments, the second angle is determined using the medial surface and lateral surface along the distal tip length. In some embodiments, a third angle between the first distal tip edge and the second distal tip edge of the distal tip is less than 90 degrees. In some embodiments, the first portion comprises a medial surface connected to a lateral surface via at least two connecting surfaces, wherein at least one connecting surface of the device is a beveled surface. In some embodiments, the two connecting surfaces of the device are beveled surfaces.

Provided herein is a device for performing a medical procedure such as cutting tissue of a subject, the device comprising: a cutting head comprising a distal tip, a proximal end and a first portion comprising a first sharp edge between the distal tip and the proximal end, and an oscillator operatively coupled to the proximal end of the cutting head, wherein the cutting head has a medial surface, a lateral surface, a first connecting surface coupling the medial surface to the lateral surface, and a second connecting surface coupling the medial surface to the lateral surface. In some embodiments, the first sharp edge is at the intersection of the medial surface and the first connecting surface, at the intersection of the medial surface and the second connecting surface, at the intersection of the lateral surface and the first connecting surface, at the intersection of the lateral surface and the second connecting surface, is part of the first connecting surface, or is part of the second connecting surface. In some embodiments, the first sharp edge is substantially aligned with the medial surface of the cutting head or with the lateral surface of the cutting head. In some embodiments, the first sharp edge is not substantially aligned with the medial surface of the cutting head or with the lateral surface of the cutting head. In some embodiments, substantially aligned means that a distance from the medial surface or lateral surface to the first sharp edge is less than 2%, less than 5%, less than 10%, or less than 20% of a thickness of the cutting head measured perpendicularly in the transverse cross-section from the surface. In some embodiments, the first connecting surface is a beveled surface. In some embodiments, the second connecting surface is a beveled surface. In some embodiments, the first portion comprises a second sharp edge. In some embodiments, the second sharp edge is at the intersection of the medial surface and the first connecting surface, at the intersection of the medial surface and the second connecting surface, at the intersection of the lateral surface and the first connecting surface, at the intersection of the lateral surface and the second connecting surface, is part of the first connecting surface, or is part of the second connecting surface. In some embodiments, the first portion of the cutting head comprises a trapezoidal transverse cross-section. In some embodiments, the connecting surface (e.g., beveled surface) forms an angle of from 0 degrees to 30 degrees, from 30 degrees to 45 degrees, from 0 degrees to 45 degrees, from 45 degrees to 90 degrees, from 45 degrees to 60 degrees, from 30 degrees to 60 degrees with the medial surface of the cutting head. In some embodiments, the connecting surface (e.g., beveled surface) forms an angle of from 0 degrees to 30 degrees, from 30 degrees to 45 degrees, from 0 degrees to 45 degrees, from 45 degrees to 90 degrees, from 45 degrees to 60 degrees, from 30 degrees to 60 degrees with the medial surface of the cutting head. In some embodiments, the first sharp edge is at the intersection of the medial surface and the connecting surface (e.g., beveled surface). In some embodiments, the connecting surface forming the first sharp edge is a beveled surface, and wherein first sharp edge is formed by an angle between the medial surface and the connecting surface (e.g., beveled surface) of between 0 degrees and 90 degrees, from 90 degrees to less than 180 degrees, from 90 degrees to 135 degrees, or from 135 degrees to less than 180 degrees with the lateral surface of the cutting head. In some embodiments, the connecting surface (e.g., beveled surface) forms an angle of from 0 degrees to 30 degrees, from 30 degrees to 45 degrees, from 0 degrees to 45 degrees, from 45 degrees to 90 degrees, from 45 degrees to 60 degrees, from 30 degrees to 60 degrees with the lateral surface of the cutting head. In some embodiments, the first sharp edge is at the intersection of the lateral surface and the connecting surface (e.g., a beveled surface). In some embodiments, the first sharp edge is at the intersection of the medial surface and the first connecting surface, at the intersection of the medial surface and the second connecting surface, at the intersection of the lateral surface and the first connecting surface, at the intersection of the lateral surface and the second connecting surface, is part of the first connecting surface, or is part of the second connecting surface. In some embodiments, the first portion is curved. In some embodiments, the first portion has a radius of curvature of at least 1 mm, at least 2 mm, at least 3 mm, at least 4 mm, at least 5 mm, at least 6 mm, at least 7 mm, at least 8 mm at least 9 mm, at least 10 mm, more than 10 mm, from 1 mm to 10 mm, from 2 mm to 9 mm, from 2 mm to 8 mm, from 2 mm to 7 mm, from 2 mm to 6 mm, from 2 mm to 5 mm, from 2 mm to 4 mm, or from 2 mm to 3 mm. In some embodiments, the curved portion comprises a radius of curvature of 2.75 mm. In some embodiments, the first portion is semicircular. In some embodiments, the distal tip is needle-shaped.

Provided herein is a device for performing a medical procedure such as cutting tissue of a subject, the device comprising: a cutting head comprising a distal tip, a first portion, and a proximal end, the first portion comprising a first sharp edge; an oscillator operatively coupled to the proximal end of the cutting head; and a plurality of actuator controls, each actuator control configured to operate the oscillator. In some embodiments, a first actuator control of the plurality of actuator controls is positioned on a housing of the device from 45 degrees to 180 degrees apart from a second actuator control of the plurality of actuator controls, measured in a circumferential arc about a longitudinal axis of the device. In some embodiments, the first actuator control of the plurality of actuator controls is positioned on the housing about 90 degrees apart from the second actuator control, measured in a circumferential arc about the longitudinal axis of the device. In some embodiments, the first actuator control of the plurality of actuators is positioned on the housing about 180 degrees apart from the second actuator control, measured in a circumferential arc about the longitudinal axis of the device. In some embodiments, the first actuator control of the plurality of actuator controls is positioned on the housing about 90 degrees apart from the medial surface of the handle of the cutting head, measured in a circumferential arc about the longitudinal axis of the device. In some embodiments, the oscillator moves the first sharp edge back and forth in a direction substantially parallel to a medial surface or a lateral surface of a handle of the cutting head, thereby moving the first sharp edge into tissue and away from tissue during use. In some embodiments, the oscillator moves the first sharp edge back and forth in a direction substantially along a line intersecting the transverse and coronal planes of the device, thereby moving the first sharp edge into a subject and away from a subject during use. In some embodiments, the oscillator moves the first sharp edge back and forth in a direction substantially along the longitudinal axis of the device. In some embodiments, the oscillator minimizes the perpendicular movement of the first sharp edge back and forth in a direction of a line intersecting the transverse and sagittal planes of the device such that the perpendicular movement of the first sharp edge is about 50% or less, less than 40% of, or less than 20% of as compared to the movement distance in the direction substantially parallel to the line intersecting the transverse and coronal planes of the device. In some embodiments, the oscillator moves the first sharp edge at a rate of at least 300 Hz, at least 400 Hz, at least 500 Hz, at least 1000 Hz, at least 1500 Hz, at least 2000 Hz, at least 2500 Hz, at least 3000 Hz, at least 3500 Hz, at least 4000 Hz, at least 4500 Hz, or at least 5000 Hz. In some embodiments, the oscillator is configured to oscillate the cutting head at a rate of at least 100 Hz, at least 300 Hz, at least 1000 Hz, at least 3000 Hz, or at least 5000 Hz. In some embodiments, the device is further comprising a rotational actuation control configured to rotate the cutting head about a longitudinal axis of the device. The rotational actuation may occur while the oscillator is active, thereby rotating the blade while cutting by oscillation or vibration. In some embodiments, the device further comprises a cauterization feature which delivers energy to the cutting head or a portion thereof (e.g. the tip, the first blade, the second blade, or any combination thereof) to cauterize the tissue simultaneously with cutting or after the tissue is cut by the device, and/or simultaneously with rotation, or before or after the head is rotated.

A device for performing a medical procedure such as cutting tissue of a subject, the device comprising: a cutting head comprising a distal tip, a proximal end, a first portion comprising a first sharp edge positioned adjacent the distal tip, and a handle positioned proximal to the first portion and adjacent to the proximal end; and a cutting device body having an oscillator housed therein and a handle connector configured to couple the oscillator to the handle of cutting head. In some embodiments, the handle connector irreversibly couples the cutting head to the cutting device body. In some embodiments, the handle connector comprises one or more of a chemical fixture, a mechanical fixture, or a friction joint. In some embodiments, the handle connector includes an opening with an inner dimension substantially sized to fit the handle thickness therein, and includes a handle fixture flange extending from the wall at a distal end of the handle connector and into the opening of the connector. In some embodiments, the cutting head comprises a handle fixture notch on the handle configured to align with the handle fixture flange in position and size when inserted in the handle connector opening. In some embodiments, when inserted into the device, the handle deflects the fixture flange away from the longitudinal axis until the fixture flange reaches the notch, which prevents reverse movement of the handle out of the handle connector. In some embodiments, the handle comprises metal or stiff plastic having a Young's modulus greater than 3.0 GPa at room temperature and the handle connector comprises metal or stiff plastic having a Young's modulus greater than 3.0 GPa at room temperature, thereby generating a metal-to-metal interface, a metal-to-plastic interface, or a plastic-to plastic interface. In some embodiments, the handle configured to outer dimensions are within 1%, 2%, 3%, 5%, 10%, <10%, <8%, <5%, <2%, <1%, <0.5%, <0.2%, 0.5 to 5%, 0.5 to 2%, 0.1 to 5%, or 0.1 to 10% of the inner dimensions of the handle connector. In some embodiments, one or both of the handle connector and the handle comprise one or more alignment guides configured to ensure the handle inserts in the proper orientation relative to the cutting device body and the handle connector. In some embodiments, one or both of the handle connector and the handle comprise one or more an insertion distance guides that provide visual or audible indication of proper insertion and fixation of the handle in the device body. In some embodiments, at least a portion of the cutting head has a thickness of 0.4 mm to 0.6 mm. In some embodiments, at least a portion of the cutting head has a thickness of 0.5 mm. In some embodiments, the rotational actuation control comprises a lock. In some embodiments, the oscillator is configured to oscillate the cutting head at a rate of at least 100 Hz, at least 300 Hz, at least 1000 Hz, at least 3000 Hz, or at least 5000 Hz.

Provided herein is device of any embodiment described herein comprising a cauterization energy source coupled to the cutting head configured to cauterize the tissue cut by the blade of the device by delivering cauterization energy to the first sharp edge of the cutting head. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A and FIG. 1B show a portion of a cutting device for performing medical procedures, in accordance with embodiments; FIG. 1C shows rotation of a cutting head of a device for performing medical procedures, in accordance with embodiments; FIG. 1D shows a cross-sectional schematic of a cutting device, in accordance with embodiments; FIG. 1E shows an external view of a cutting device, in accordance with embodiments; FIG. 1F shows an external view of a cutting device, in accordance with embodiments; FIG. 1G shows an external view of a cutting device, in accordance with embodiments; FIG. 1H shows an external view of a cutting device, in accordance with embodiments; FIG. 1I shows an external view of a cutting device, in accordance with embodiments; FIG. 1J shows a schematic of a cutting device, in accordance with embodiments.

FIG. 2A shows a frontal view of a wire useful in the formation of a cutting head, in accordance with embodiments; FIG. 2B shows a side view of the wire shown in FIG. 2A, in accordance with embodiments. FIG. 2C shows a frontal view of a cutting head comprising a curved region, in accordance with embodiments; FIG. 2D show a side view of the cutting head shown in FIG. 2C having a shoulder region, in accordance with embodiments. FIGS. 2E-2H show various exemplary embodiments of a cutting head, in accordance with embodiments.

FIGS. 3A-3S show transverse cross-sections of a cutting head, in accordance with embodiments.

FIGS. 4A-4E show sagittal cross-sections of a cutting head, in accordance with embodiments. FIGS. 4F-4L show frontal views of a cutting head, in accordance with embodiments.

FIG. 5 shows an exemplary embodiment of the use of a cutting device in an eye procedure.

FIG. 6A-6C show exemplary embodiments of cuts in a tissue that can be made during an eye procedure using a cutting device, in accordance with embodiments.

FIG. 7A shows a portion of a cutting head handle comprising a handle fixture notch, in accordance with embodiments; FIG. 7B shows a handle connector comprising a handle fixture flange, in accordance with embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure is described in relation to devices, systems, and methods for medical procedures, such as eye procedures, which include cataract surgery. However, one of skill in the art will appreciate that this is not intended to be limiting and the devices and methods disclosed herein may be used in other anatomical areas and in other surgical procedures.

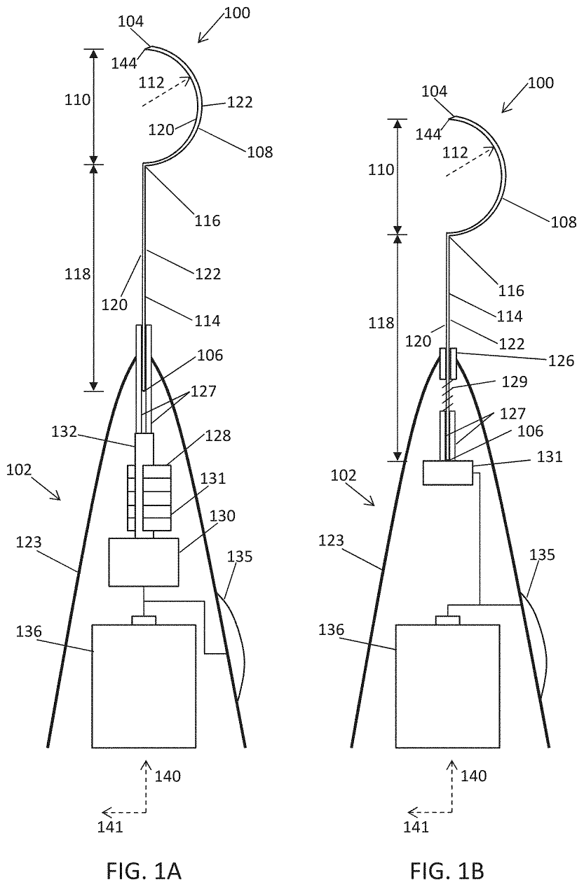

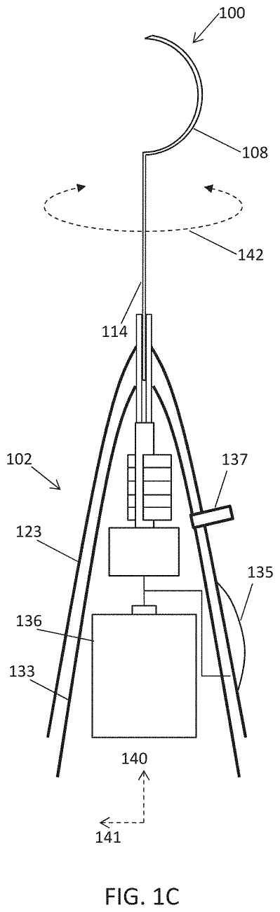

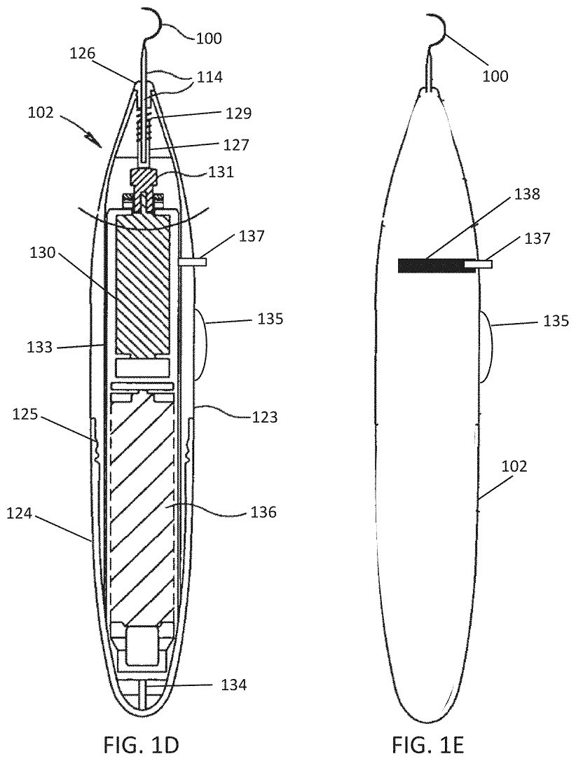

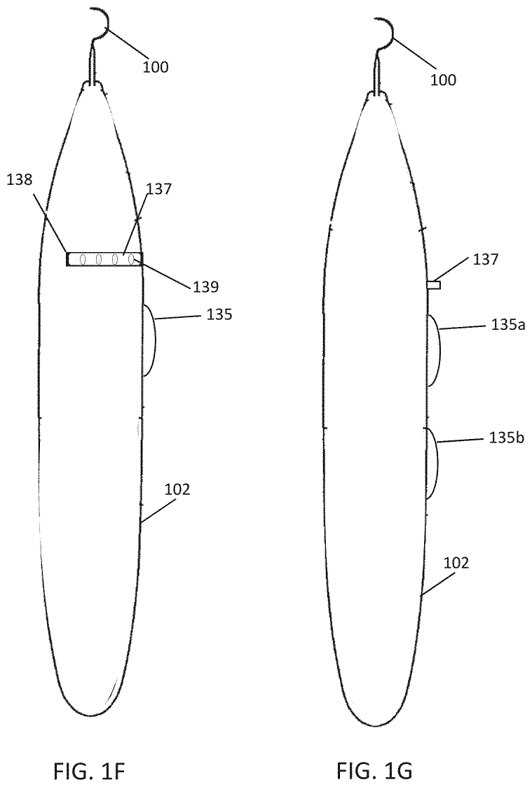

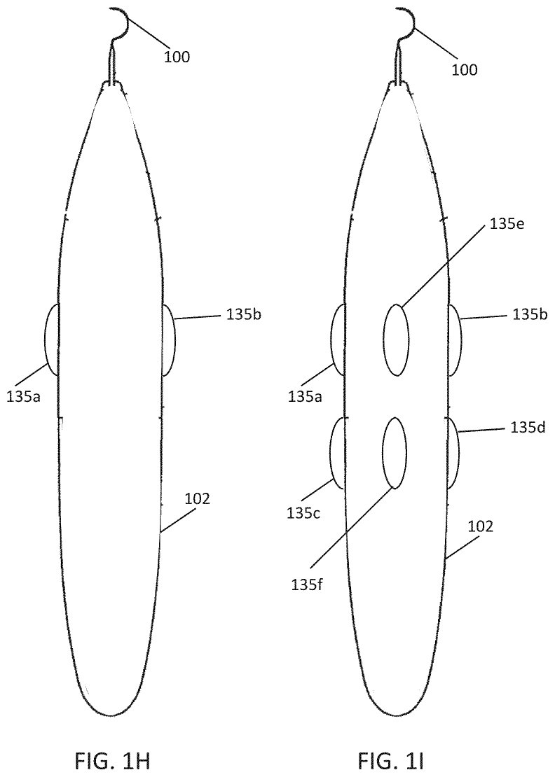

FIGS. 1A and 1B show cross-sectional schematics of exemplary embodiments of cutting devices, which can be used to during ophthalmic procedures (e.g., eye procedures, such as cataract surgery). In some embodiments, a cutting device includes a cutting head 100 connected through a cutting head handle 114 or the proximal end 106, or both, to a cutting device body 102. In some embodiments, a cutting head 100 of a cutting device comprises a distal tip 104, a first portion 108, a proximal end 106, and a handle 114. A first portion 108 comprises an edge in many embodiments. In many embodiments, a first portion 108 comprises a plurality of edges. For example, a first portion 108 of cutting head 100 comprises a first edge (e.g., edge 332) and a second edge (e.g., edge 334), in many embodiments. In many embodiments, cutting head 100 comprises a blade portion comprising at least one sharp edge. In some embodiments, cutting head 100 comprises a blade portion comprising one sharp edge and one blunt edge. In some embodiments, cutting head 100 comprises a blade portion comprising two sharp edges. In some embodiments, all or a portion of first portion 108 of cutting head 100 comprises a sharp edge (e.g., edge 332, 332a, 332b, 334, 334a, or 334b). In some embodiments, all or a portion of first portion 108 of cutting head 100 comprises a plurality of sharp edges. For example, all or a portion of first portion 108 of cutting head 100 comprises a first sharp edge (e.g., edge 332) and a second sharp edge (e.g., 334), in many embodiments. In some embodiments, all or a portion of first portion 108 of cutting head 100 comprises exactly one sharp edge and at least one non-sharp (e.g., blunted or rounded) edge (e.g., edge 333, 333a, 333b, 335, 335a, or 335b). In some embodiments, all or a portion of first portion 108 of cutting head 100 comprises 1, 2, 3, 4, 5, 6, or more than 6 non-sharp (e.g., blunted or rounded) edges. A sharp edge of cutting head 100 can be useful in making an incision or cut into a tissue of a subject, including one or more tissues of an eye (e.g., a capsule or lens of an eye). In some embodiments, a cutting head 100 comprises a pointed and/or sharp tip. In some embodiments, distal tip 104 of cutting head 100 comprises a sharp point or sharp edge. A pointed and/or sharp tip of cutting head 100 can aid in making an incision or cut into a tissue of a subject, including one or more tissues of an eye (e.g., a capsule or lens of an eye). In some embodiments, cutting head 100 comprises a medial surface 120 and a lateral surface 122. In some embodiments, a medial surface 120 is closer to a longitudinal axis 140 of the cutting device than a lateral surface 122, as measured in a radial direction 141.

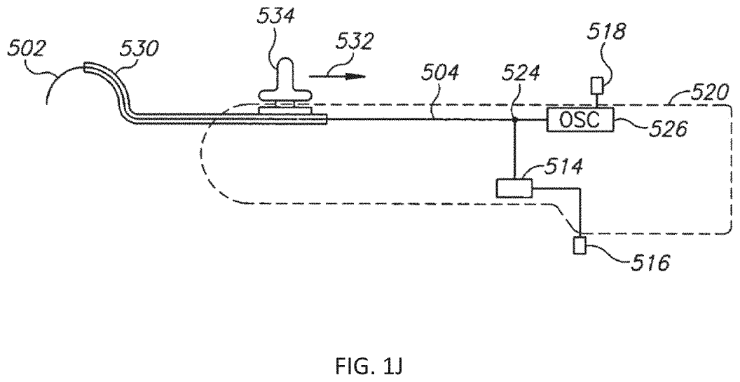

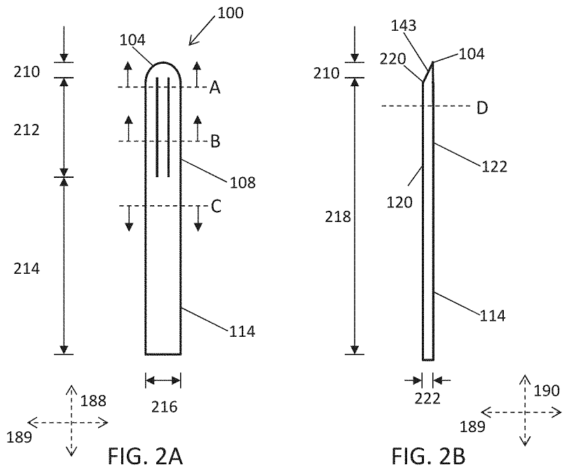

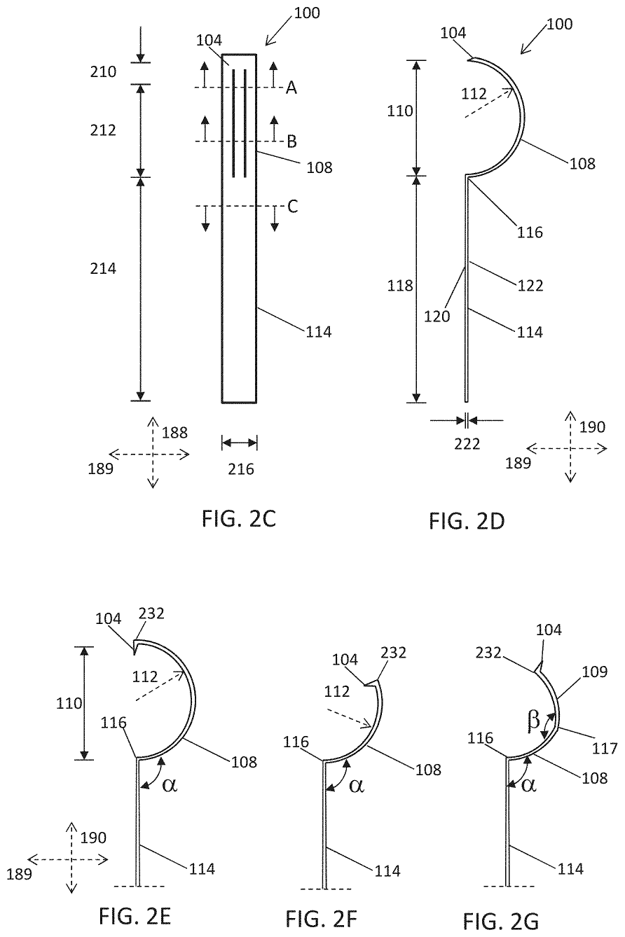

All or a portion of cutting head 100 can be curved. In some embodiments, all or a portion of first portion 108 comprises a curve. In some embodiments, curved portion of cutting head 100 comprises a sharp edge and/or a pointed tip. In some embodiments, a curved portion of cutting head 100 comprises a plurality of sharp edges (e.g., 2 sharp edges or more than 2 sharp edges). In many embodiments, curved portion of cutting head 100 (e.g., all or a portion of first portion 108) has a radius of curvature 112. In some embodiments, a curve of cutting head 100 comprises a circular arc. In some embodiments, a curve of cutting head 100 comprises an elliptical arc. In some embodiments, a curve of cutting head 100 comprises an ellipsoid arc. In some embodiments, a curve of cutting head 100 comprises an oval arc. In some embodiments, a curve of cutting head 100 comprises an ovoid arc. In some embodiments, a curve of cutting head 100 comprises a variable radius arc that is non-circular. In some embodiments, a curve of cutting head 100 comprises a non-straight path along its length from the proximal end to the tip of the cutting head. In some embodiments, a curve of cutting head 100 comprises a circular arc of less than 360 and more than 0 degrees. In some embodiments, cutting head 100 comprises a circular arc of 30 degrees to 270 degrees. In some embodiments, a cutting head (or a portion thereof, such as all or a portion of first portion 108) comprises a circular arc of 30 degrees to 45 degrees, 30 degrees to 60 degrees, 30 degrees to 90 degrees, 30 degrees to 180 degrees, 30 degrees to 215 degrees, 30 degrees to 225 degrees, 30 degrees to 270 degrees, 45 degrees to 60 degrees, 45 degrees to 90 degrees, 45 degrees to 180 degrees, 45 degrees to 215 degrees, 45 degrees to 225 degrees, 45 degrees to 270 degrees, 60 degrees to 90 degrees, 60 degrees to 180 degrees, 60 degrees to 215 degrees, 60 degrees to 225 degrees, 60 degrees to 270 degrees, 90 degrees to 180 degrees, 90 degrees to 215 degrees, 90 degrees to 225 degrees, 90 degrees to 270 degrees, 180 degrees to 215 degrees, 180 degrees to 225 degrees, 180 degrees to 270 degrees, 215 degrees to 225 degrees, 215 degrees to 270 degrees, or 225 degrees to 270 degrees. In some embodiments, cutting head 100 or portion thereof comprises a circular arc of 30 degrees, 45 degrees, 60 degrees, 90 degrees, 180 degrees, 215 degrees, 225 degrees, or 270 degrees. In some embodiments, cutting head or portion thereof comprises a circular arc of at least 30 degrees, 45 degrees, 60 degrees, 90 degrees, 180 degrees, 215 degrees, or 225 degrees. In some embodiments, a cutting head 100 or portion thereof comprises a circular arc of at most 45 degrees, 60 degrees, 90 degrees, 180 degrees, 215 degrees, 225 degrees, or 270 degrees. In many embodiments, a curve of cutting head 100 (or a portion thereof, such as all or a portion of first portion 108) comprises a circular arc of 30 to 270 degrees, 45 degrees to 225 degrees, 60 degrees to 215 degrees, or 90 degrees to 180 degrees (e.g., as shown in FIG. 2D, FIG. 2E, FIG. 2F, and FIG. 2G). In some embodiments, a curved portion of cutting head 100 (e.g., all or a portion of first portion 108) comprises a circular arc of exactly 180 degrees. In some embodiments, a curved portion of cutting head 100 (e.g., all or a portion of first portion 108) has a diameter of 1 mm to 10 mm. In some embodiments, a curved portion of cutting head 100 (e.g., all or a portion of first portion 108) has a diameter of 1 mm to 2 mm, 1 mm to 3 mm, 1 mm to 4 mm, 1 mm to 5 mm, 1 mm to 6 mm, 1 mm to 7 mm, 1 mm to 8 mm, 1 mm to 9 mm, 1 mm to 10 mm, 2 mm to 3 mm, 2 mm to 4 mm, 2 mm to 5 mm, 2 mm to 6 mm, 2 mm to 7 mm, 2 mm to 8 mm, 2 mm to 9 mm, 2 mm to 10 mm, 3 mm to 4 mm, 3 mm to 5 mm, 3 mm to 6 mm, 3 mm to 7 mm, 3 mm to 8 mm, 3 mm to 9 mm, 3 mm to 10 mm, 4 mm to 5 mm, 4 mm to 6 mm, 4 mm to 7 mm, 4 mm to 8 mm, 4 mm to 9 mm, 4 mm to 10 mm, 5 mm to 6 mm, 5 mm to 7 mm, 5 mm to 8 mm, 5 mm to 9 mm, 5 mm to 10 mm, 6 mm to 7 mm, 6 mm to 8 mm, 6 mm to 9 mm, 6 mm to 10 mm, 7 mm to 8 mm, 7 mm to 9 mm, 7 mm to 10 mm, 8 mm to 9 mm, 8 mm to 10 mm, or 9 mm to 10 mm. In some embodiments, a curved portion of cutting head 100 (e.g., all or a portion of first portion 108) has a diameter of 1 mm, 2 mm, 3 mm, 4 mm, 5 mm, 6 mm, 7 mm, 8 mm, 9 mm, or 10 mm. In some embodiments, a curved portion of cutting head 100 (e.g., all or a portion of first portion 108) has a diameter of at least 1 mm, 2 mm, 3 mm, 4 mm, 5 mm, 6 mm, 7 mm, 8 mm, or 9 mm. In some embodiments, a curved portion of cutting head 100 (e.g., all or a portion of first portion 108) has a diameter of at most 2 mm, 3 mm, 4 mm, 5 mm, 6 mm, 7 mm, 8 mm, 9 mm, or 10 mm. In some embodiments, a curved portion of cutting head 100 has a diameter of 5.5 mm.

In some embodiments, a handle of cutting head 100 has a length 118 of 1 mm to 12 mm. In some embodiments, a handle of cutting head 100 has a length of 1 mm to 2 mm, 1 mm to 3 mm, 1 mm to 4 mm, 1 mm to 5 mm, 1 mm to 6 mm, 1 mm to 7 mm, 1 mm to 8 mm, 1 mm to 9 mm, 1 mm to 10 mm, 1 mm to 11 mm, 1 mm to 12 mm, 2 mm to 3 mm, 2 mm to 4 mm, 2 mm to 5 mm, 2 mm to 6 mm, 2 mm to 7 mm, 2 mm to 8 mm, 2 mm to 9 mm, 2 mm to 10 mm, 2 mm to 11 mm, 2 mm to 12 mm, 3 mm to 4 mm, 3 mm to 5 mm, 3 mm to 6 mm, 3 mm to 7 mm, 3 mm to 8 mm, 3 mm to 9 mm, 3 mm to 10 mm, 3 mm to 11 mm, 3 mm to 12 mm, 4 mm to 5 mm, 4 mm to 6 mm, 4 mm to 7 mm, 4 mm to 8 mm, 4 mm to 9 mm, 4 mm to 10 mm, 4 mm to 11 mm, 4 mm to 12 mm, 5 mm to 6 mm, 5 mm to 7 mm, 5 mm to 8 mm, 5 mm to 9 mm, 5 mm to 10 mm, 5 mm to 11 mm, 5 mm to 12 mm, 6 mm to 7 mm, 6 mm to 8 mm, 6 mm to 9 mm, 6 mm to 10 mm, 6 mm to 11 mm, 6 mm to 12 mm, 7 mm to 8 mm, 7 mm to 9 mm, 7 mm to 10 mm, 7 mm to 11 mm, 7 mm to 12 mm, 8 mm to 9 mm, 8 mm to 10 mm, 8 mm to 11 mm, 8 mm to 12 mm, 9 mm to 10 mm, 9 mm to 11 mm, 9 mm to 12 mm, 10 mm to 11 mm, 10 mm to 12 mm, or 11 mm to 12 mm, 13 mm to 14 mm, 14 mm to 15 mm, 15 mm to 16 mm, 16 mm to 17 mm, 17 mm to 18 mm, 18 mm to 19 mm, 19 mm to 20 mm, 1 mm to 20 mm, 3 mm to 17 mm, 5 mm to 15 mm, 7 mm to 13 mm, or 9 mm to 11 mm. In some embodiments, a handle of cutting head 100 has a length of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, a handle of cutting head 100 has a length of at least 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, a handle of cutting head 100 has a length of at most 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, a curved portion of cutting head 100 is separated from a straight (e.g., flat) portion of cutting head 100 (e.g., handle 114) by a bend 116. Bend 116 comprises an angle alpha (e.g., □, as shown in FIGS. 2E-2G), in some embodiments. In some embodiments, (e.g., as shown in FIG. 2G), a cutting head 100 comprises a plurality of bends (e.g., first bend 116, second bend 117, and/or bend 232, for example, as shown in FIGS. 2E-2G). In some embodiments, a bend separates a first portion (e.g., curved portion) and a second portion (e.g., curved portion) of a cutting head 100. In some embodiments, bend 117 divides a first portion 108 of a cutting head and a second portion 109 of a cutting head. In some embodiments, bend 117 comprises an angle beta (e.g., □, as shown in FIG. 2G). In some embodiments, a bend of cutting head 100 (e.g., bend 116, bend 117, and/or bend 232) comprises an angle of 0 degrees to 30 degrees, 0 degrees to 45 degrees, 0 degrees to 60 degrees, 0 degrees to 90 degrees, 0 degrees to 135 degrees, 0 degrees to 180 degrees, 30 degrees to 45 degrees, 30 degrees to 60 degrees, 30 degrees to 90 degrees, 30 degrees to 135 degrees, 30 degrees to 180 degrees, 45 degrees to 60 degrees, 45 degrees to 90 degrees, 45 degrees to 135 degrees, 45 degrees to 180 degrees, 60 degrees to 90 degrees, 60 degrees to 135 degrees, 60 degrees to 180 degrees, 90 degrees to 135 degrees, 90 degrees to 180 degrees, or 135 degrees to 180 degrees. In some embodiments, bend 116 comprises an angle of 0 degrees, 30 degrees, 45 degrees, 60 degrees, 90 degrees, 135 degrees, or 180 degrees. In some embodiments, bend 116 comprises an angle of at least 0 degrees, 30 degrees, 45 degrees, 60 degrees, 90 degrees, or 135 degrees. In some embodiments, bend 116 comprises an angle of at most 30 degrees, 45 degrees, 60 degrees, 90 degrees, 135 degrees, or 180 degrees.

In many embodiments, a cutting head 100 (or portion thereof) is connected to (e.g., coupled to) an oscillator 128. In many embodiments, an oscillator 128 is configured to move (e.g., oscillate or vibrate) cutting head 100 back and forth in an axial direction (e.g., along a longitudinal axis 140 of the cutting device), indicated by arrow 110. Alternatively or additionally, an oscillator can be configured to move (e.g., oscillate or vibrate) cutting head 100 around longitudinal axis 140 of the cutting device. Actuation of cutting head 100 can aid in the execution of eye procedure steps comprising cutting into a tissue of an eye, such as a lens of the eye. Actuation (e.g., oscillation or vibration) of all or a portion of cutting head 100 can be used to achieve a cut in the tissue in the shape of all or a portion of cutting head 100 when placed on tissue. In some embodiments, actuation of all or a portion of cutting head 100 can be useful in making an incision into a capsule of an eye and/or in making a cut into a lens of an eye (e.g., during an eye procedure, such as cataract surgery).

In many embodiments, actuation of a cutting head using an oscillator, as disclosed herein, can improve the precision with which a cut can be made in a tissue of an eye. In some embodiments, actuation of a cutting head using a device, system, or method disclosed herein can minimize damage to one or more tissues adjacent to or in the vicinity of a tissue being cut during an eye procedure. For example, actuation of a cutting head using a device, system, or method disclosed herein can minimize unintended damage to one or more tissues (e.g., of an eye) by minimizing the amount of movement a practitioner must manually impart on a cutting head by moving his or her hands or arms (e.g., in a sawing motion).

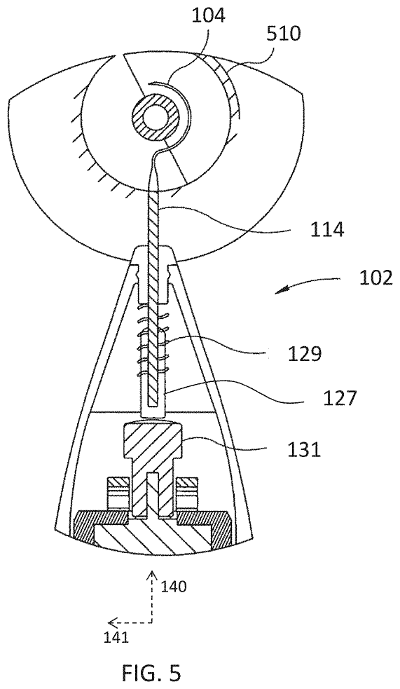

In some embodiments, an oscillator 131 of a cutting device comprises an eccentric rotating mass vibration motor (e.g., as shown in FIG. 1A). In some embodiments, an oscillator 131 of a cutting device comprises a linear resonant actuator (e.g., as shown in FIG. 1B). In some embodiments, an oscillator 131 comprises a motor 130 (e.g., oscillation motor 526). In some embodiments, motor 130 is coupled to shaft 132, for example, such that motor 130 causes shaft 132 to rotate when operated. In some embodiments, motor 130 (e.g., rotation motor 514) is configured to rotate shaft 132 about longitudinal axis 140. In some embodiments, motor 130 is configured to rotate shaft about longitudinal axis 140 at a rate of from about 100 Hz to about 5,000 Hz. In some embodiments, motor 130 is configured to rotate shaft about longitudinal axis 140 at a rate of from about 100 Hz to about 200 Hz, about 100 Hz to about 300 Hz, about 100 Hz to about 400 Hz, about 100 Hz to about 500 Hz, about 100 Hz to about 1,000 Hz, about 100 Hz to about 3,000 Hz, about 100 Hz to about 5,000 Hz, about 200 Hz to about 300 Hz, about 200 Hz to about 400 Hz, about 200 Hz to about 500 Hz, about 200 Hz to about 1,000 Hz, about 200 Hz to about 3,000 Hz, about 200 Hz to about 5,000 Hz, about 300 Hz to about 400 Hz, about 300 Hz to about 500 Hz, about 300 Hz to about 1,000 Hz, about 300 Hz to about 3,000 Hz, about 300 Hz to about 5,000 Hz, about 400 Hz to about 500 Hz, about 400 Hz to about 1,000 Hz, about 400 Hz to about 3,000 Hz, about 400 Hz to about 5,000 Hz, about 500 Hz to about 1,000 Hz, about 500 Hz to about 3,000 Hz, about 500 Hz to about 5,000 Hz, about 1,000 Hz to about 3,000 Hz, about 1,000 Hz to about 5,000 Hz, or about 3,000 Hz to about 5,000 Hz. In some embodiments, motor 130 is configured to rotate shaft about longitudinal axis 140 at a rate of from about 100 Hz, about 200 Hz, about 300 Hz, about 400 Hz, about 500 Hz, about 1,000 Hz, about 3,000 Hz, or about 5,000 Hz. In some embodiments, motor 130 is configured to rotate shaft about longitudinal axis 140 at a rate of from at least about 100 Hz, about 200 Hz, about 300 Hz, about 400 Hz, about 500 Hz, about 1,000 Hz, or about 3,000 Hz. In some embodiments, motor 130 is configured to rotate shaft about longitudinal axis 140 at a rate of from at most about 200 Hz, about 300 Hz, about 400 Hz, about 500 Hz, about 1,000 Hz, about 3,000 Hz, or about 5,000 Hz. One or more weights 128 are coupled to shaft 132, in many embodiments. In many embodiments, the center of mass of weight(s) 128 is located at a radius from longitudinal axis 140 greater than that of the rotational center of motor 130 (e.g., oscillation motor 526) and/or shaft 132. In some embodiments, such a configuration imparts an oscillatory or vibratory motion on cutting head 100 (or a portion thereof) when motor 130 is operated. In use during an eye procedure (e.g., a cataract surgery, as shown in FIG. 5), longitudinal axis 140 of the cutting device is aligned with the center of the eye lens, in many embodiments.

In many embodiments, an oscillator 131 (e.g., a linear resonant actuator, LRA) is coupled to handle 114 of cutting head 100 (e.g., via handle connector 127, which can be directly coupled to handle 114, housing 123 and/or an oscillator). In some embodiments, oscillator 131 is coupled directly to housing 123 and/or spindle 133. In some embodiments, oscillator 131 causes cutting head 100 to oscillate or vibrate when oscillator 131 is operated (e.g., by imparting an axial movement on all or a portion of cutting head 100). In some embodiments, the movement of cutting head 100 caused by oscillator 131 can be aided by including a spring 129 in a cutting device disclosed herein. For example, in some embodiments, spring 129 is disposed between (and, optionally, biased against): housing 123 and oscillator 131, housing 123 and handle connector 127, handle connector 127 and oscillator 131, handle channel 126 and handle connector 127, and/or handle channel 126 and oscillator 131, for example, to impart a resistive force to the motion of oscillator 131. In some embodiments, handle channel 126 is coupled directly to housing 123. In some embodiments, handle 114 enters housing 123 by passing through handle channel 126. In some embodiments, oscillator 131 is configured to cause cutting head 100 to oscillate or vibrate at a rate of from about 10 Hz to about 5,000 Hz. In some embodiments, oscillator 131 is configured to cause cutting head 100 to oscillate or vibrate at a rate of from about 10 Hz to about 50 Hz, about 10 Hz to about 100 Hz, about 10 Hz to about 200 Hz, about 10 Hz to about 300 Hz, about 10 Hz to about 400 Hz, about 10 Hz to about 500 Hz, about 10 Hz to about 1,000 Hz, about 10 Hz to about 2,000 Hz, about 10 Hz to about 3,000 Hz, about 10 Hz to about 4,000 Hz, about 10 Hz to about 5,000 Hz, about 50 Hz to about 100 Hz, about 50 Hz to about 200 Hz, about 50 Hz to about 300 Hz, about 50 Hz to about 400 Hz, about 50 Hz to about 500 Hz, about 50 Hz to about 1,000 Hz, about 50 Hz to about 2,000 Hz, about 50 Hz to about 3,000 Hz, about 50 Hz to about 4,000 Hz, about 50 Hz to about 5,000 Hz, about 100 Hz to about 200 Hz, about 100 Hz to about 300 Hz, about 100 Hz to about 400 Hz, about 100 Hz to about 500 Hz, about 100 Hz to about 1,000 Hz, about 100 Hz to about 2,000 Hz, about 100 Hz to about 3,000 Hz, about 100 Hz to about 4,000 Hz, about 100 Hz to about 5,000 Hz, about 200 Hz to about 300 Hz, about 200 Hz to about 400 Hz, about 200 Hz to about 500 Hz, about 200 Hz to about 1,000 Hz, about 200 Hz to about 2,000 Hz, about 200 Hz to about 3,000 Hz, about 200 Hz to about 4,000 Hz, about 200 Hz to about 5,000 Hz, about 300 Hz to about 400 Hz, about 300 Hz to about 500 Hz, about 300 Hz to about 1,000 Hz, about 300 Hz to about 2,000 Hz, about 300 Hz to about 3,000 Hz, about 300 Hz to about 4,000 Hz, about 300 Hz to about 5,000 Hz, about 400 Hz to about 500 Hz, about 400 Hz to about 1,000 Hz, about 400 Hz to about 2,000 Hz, about 400 Hz to about 3,000 Hz, about 400 Hz to about 4,000 Hz, about 400 Hz to about 5,000 Hz, about 500 Hz to about 1,000 Hz, about 500 Hz to about 2,000 Hz, about 500 Hz to about 3,000 Hz, about 500 Hz to about 4,000 Hz, about 500 Hz to about 5,000 Hz, about 1,000 Hz to about 2,000 Hz, about 1,000 Hz to about 3,000 Hz, about 1,000 Hz to about 4,000 Hz, about 1,000 Hz to about 5,000 Hz, about 2,000 Hz to about 3,000 Hz, about 2,000 Hz to about 4,000 Hz, about 2,000 Hz to about 5,000 Hz, about 3,000 Hz to about 4,000 Hz, about 3,000 Hz to about 5,000 Hz, or about 4,000 Hz to about 5,000 Hz. In some embodiments, oscillator 131 is configured to cause cutting head 100 to oscillate or vibrate at a rate of from about 10 Hz, about 50 Hz, about 100 Hz, about 200 Hz, about 300 Hz, about 400 Hz, about 500 Hz, about 1,000 Hz, about 2,000 Hz, about 3,000 Hz, about 4,000 Hz, or about 5,000 Hz. In some embodiments, oscillator 131 is configured to cause cutting head 100 to oscillate or vibrate at a rate of from at least about 10 Hz, about 50 Hz, about 100 Hz, about 200 Hz, about 300 Hz, about 400 Hz, about 500 Hz, about 1,000 Hz, about 2,000 Hz, about 3,000 Hz, or about 4,000 Hz. In some embodiments, oscillator 131 is configured to cause cutting head 100 to oscillate or vibrate at a rate of from at most about 50 Hz, about 100 Hz, about 200 Hz, about 300 Hz, about 400 Hz, about 500 Hz, about 1,000 Hz, about 2,000 Hz, about 3,000 Hz, about 4,000 Hz, or about 5,000 Hz.

An actuator control (e.g., a switch, button, dial, touchpad, gear, knob, or other control) is mounted on housing 123 of a cutting device in some embodiments. An actuator control (e.g., oscillation activation mechanism 135) is used to control the operation of oscillator 128 in many embodiments. In many embodiments, an actuator control (e.g., rotational actuation control 137) is used to control the rotation of cutting head 100 or a portion thereof around a longitudinal axis 140. In some embodiments, a cutting device comprises a plurality of actuators. For example, a first actuator control is used to control the operation of an oscillator and a second actuator control is used to control the rotation of at least a portion of the cutting device (e.g., cutting head 100) around a longitudinal axis 140 of the cutting device in some embodiments. As described herein, a cutting device comprises a plurality of oscillation activation mechanisms 135, in some embodiments (e.g., as shown in FIGS. 1G-1I). For example, a cutting device comprises 1, 2, 3, 4, 5, 6, 7, 8, 9, 10, or more than 10 oscillation activation mechanisms 135, in some embodiments. In some embodiments, a cutting device described herein comprises a plurality of rotational actuation controls 137. Advantages of a cutting device comprising a plurality of oscillation activation mechanisms 135 include ease of access to oscillation controls when the housing of the cutting device is rotated in a user's hand (e.g., to manually rotate cutting head 100 relative to a biological tissue, such as an eye) and the ability to include dedicated actuators for each function of the oscillator (e.g., a first button for activation and deactivation and a second button for controlling oscillation speed).

In some embodiments, a cutting device disclosed herein comprises 1, 2, 3, 4, 5, 6, 7, 8, 9, 10, or more than 10 actuators (e.g., oscillation activation mechanisms 135 or rotational actuation controls 137, for example, comprising a button, switch, touchpad, lever, or a combination thereof). A cutting device disclosed herein comprises 1, 2, 3, 4, 5, 6, 7, 8, 9, 10, or more than 10 oscillation activation mechanisms (e.g., oscillation activation mechanisms 135, 135a, 135b, 135c, 135d, 135e, and/or 135f) configured to operate an oscillator. In some embodiments, a cutting device comprises one or more actuation controls (e.g., oscillation activation mechanism 135, 135a, 135b, 135c, 135d, 135e, and/or 135f) configured to activate an oscillator 131. In some embodiments, a cutting device comprises one or more actuation controls (e.g., oscillation activation mechanism 135, 135a, 135b, 135c, 135d, 135e, and/or 135f) configured to deactivate an oscillator. In some embodiments, an actuation control of a cutting device is configured to activate an oscillator based on a first set of one or more actuations (e.g., wherein a set of one or more actuations comprises a single actuation or a plurality of actuations of the actuation control) and to deactivate an oscillator based on a second set of one or more actuations (e.g., wherein a set of one or more actuations comprises a single actuation or a plurality of actuations of the actuation control).

In some embodiments, a cutting device comprises a plurality of oscillation activation mechanisms 135 configured to activate an oscillator of the cutting device (e.g., as shown in FIG. 1G, FIG. 1H, and FIG. 1I). A cutting device comprises two oscillation activation mechanisms 135 configured to activate an oscillator of the cutting device, in some embodiments. In some embodiments, a first oscillation activation mechanism 135 is located at a first location on the body of the cutting device and a second oscillation activation mechanism is located at a second location on the body of the cutting device (e.g., as shown in FIG. 1H and FIG. 1I). A cutting device comprises two oscillation activation mechanisms 135 configured to deactivate an oscillator of the cutting device, in some embodiments. In some embodiments, the second location is spaced from zero degrees to 30 degrees, from 30 degrees to 60 degrees, from 60 degrees to 90 degrees, from 90 degrees to 120 degrees, or from 120 degrees to 180 degrees from the first location, as measured circumferentially around the cutting device's body (e.g., around longitudinal axis 140). For example, in some embodiments, an oscillation activation mechanism configured to activate an oscillator of a cutting device is located at the same circumferential position around arc 142 as an oscillation activation mechanism configured to deactivate the oscillator. In some embodiments, an oscillation activation mechanism configured to activate an oscillator of a cutting device is located at a circumferential position 180 degrees around arc 142 from a second oscillation activation mechanism configured to activate the oscillator and/or an oscillation activation mechanism configured to deactivate the oscillator. By positioning one or more oscillation activation mechanism 135 at a different position (e.g., 180 degrees) around the circumference of the cutting device from a first oscillation activation mechanism (e.g., as shown in FIG. 1H and FIG. 1I), convenient access to controls configured to activate and/or deactivate an oscillator is provided, even if a user rotates the cutting device in his or her hand (e.g., to manually rotate cutting head 100 or a portion thereof relative to a tissue of a patient, such as an eye tissue). For example, FIG. 1I shows a plurality of optional actuator controls (135a, 135b, 135c, 135d, 135e, and 135f) in exemplary positions on a housing of a cutting device disclosed herein. While FIG. 1I shows actuator controls 135a and 135c at circumferential positions on device body 102 that are 90 degrees from actuator controls 135e and 135f and 180 degrees from actuators 135b and 135d, it is contemplated that a first actuator control can be located at a position from zero degrees to 30 degrees, from 30 degrees to 60 degrees, from 60 degrees to 90 degrees, from 90 degrees to 120 degrees, or from 120 degrees to 180 degrees from the first location, as measured circumferentially around the cutting device's body (e.g., around longitudinal axis 140). For example, actuators 135a and 135b of the cutting device shown in FIG. 1G are located at positions that are separated by zero degrees, as measured circumferentially around the cutting device's body. In FIG. 1H, actuators 135a and 135b are shown at positions separated by 180 degrees, as measured circumferentially around the cutting device's body. In many embodiments, one or more of actuators 135, 135a, 135b, 135c, 135d, 135e, and/or 135f are configured to activate and/or deactivate an oscillator 131.

A cutting device also comprises a mechanism for rotating cutting head 100 (or a portion thereof) around longitudinal axis 140, in some embodiments. For example, a curved portion of cutting head 100 (e.g., first portion 108) can be rotated around longitudinal axis 140 in an arc 142, in some embodiments. In some embodiments, the oscillation or vibration continues as the cutting head 100 is rotated around the longitudinal axis 140, thereby able to cut tissue as the head 100 rotates about axis 140. In some embodiments having cauterization features included therein, either or both oscillation (or vibration) and cauterization features are able to be engaged and active during rotation of the cutting head 100 (or a portion thereof) around longitudinal axis 140.

The actuation (e.g., rotation) of a curved portion of cutting head 100 around an axis (e.g., longitudinal axis 140) can be controlled by rotational actuation control 137, for example, as shown in FIG. 1C, FIG. 1D, FIG. 1E, and FIG. 1F. In some embodiments, rotational actuation control is coupled directly to a component that is coupled directly or indirectly to cutting head 100. For example, rotational actuation control 137 is coupled to spindle 133 and/or handle connector 127 in some embodiments. In some embodiments, physically rotating all or a portion of rotational actuation control 137 in a circumferential direction about a cutting device (e.g., relative to a longitudinal axis 140) causes cutting head 100 and, optionally, one or more internal components of the cutting device to rotate in a circumferential direction about the cutting device (e.g., relative to longitudinal axis 140). In some embodiments, rotational actuation control 137 operates a mechanism (e.g., a motorized mechanism) that is configured to rotate cutting head 100 in one or two directions about longitudinal axis 140.

In some embodiments, rotational actuation control 137 comprises a motorized mechanism for rotating cutting head 100 about a longitudinal axis 140. In some embodiments, operation of rotational actuation control 137 causes one or more internal components of a cutting device (e.g., spindle 133 and/or handle connector 127) to move (e.g., rotate about longitudinal axis 140). In some embodiments, rotational actuation control 137 comprises a switch, button (e.g., button 516), or touchpad configured to operate a motor (e.g., rotation motor 514) for rotating cutting head 100 and, optionally, one or more internal components of a cutting device.

In some embodiments, a rotational actuation control 137 comprising a switch, button, touchpad, or the like is configured to rotate a cutting head in an arc of a defined length along a circumferential path 142 when contacted discretely. For example, a rotational actuation control 137 can be configured to rotate cutting head 100 (and, optionally one or more internal components of the cutting device) 15 degrees, 30 degrees, 45 degrees, 60 degrees, 75 degrees, 90 degrees, 105 degrees, 120 degrees, 135 degrees, 150 degrees, 165 degrees, 180 degrees, 225 degrees, 270 degrees, 315 degrees, 360 degrees, or along an arc defined by an angle between any two of those values when touched once or when contacted in a pattern of a plurality of touches. For example, a rotational actuation control can be configured to cause cutting head 100 (and, optionally, one or more internal components of the cutting device) to rotate in an arc of a first distance around longitudinal axis 140 on a single touch of rotational actuation control 137 and in an arc of a second distance around longitudinal axis 140 when contacted by a double touch of rotational actuation control 137. In some embodiments, rotational actuation control 137 is configured such that two or more patterns of contacts to rotational actuation control 137 cause cutting head 100 to rotate in a corresponding number of different arcs around longitudinal axis 140. In some embodiments, rotational actuation control 137 is configured to rotate cutting head 100 continuously for as long as rotational actuation control 137 is operated (e.g., contacted by a user).

In some embodiments, rotational actuation control 137 is configured to change the direction in which cutting head 100 rotates when rotational actuation control 137 is operated. For example, rotational actuation control 137 can be configured to rotate cutting head 100 in a first direction (e.g., counter-clockwise) about a circumferential arc 142 when operated in a first mode and in a second direction (e.g., clockwise) about the circumferential arc 142 when operated in a second mode. In some embodiments, rotational control 137 can be switched from the first mode to the second mode and, optionally, from the second mode to the first mode in various ways, such as by contacting rotational actuation control 137 with a pattern of one or more touches or by manually translating rotational control 137 from a first position to a second position (e.g., to engage a different gear via which rotational actuation control 137 controls rotation of cutting head 100 or simply changes a direction in which rotational actuation control 137 or a portion thereof moves).

Rotational actuation control 137 comprises a scroll wheel, in some embodiments (e.g., as shown in FIG. 1F). In some embodiments, a scroll wheel protrudes partially from housing 123 of a cutting device, allowing a user to manually rotate a cutting head 100 of the cutting device. For example, a practitioner can rotate the scroll wheel of rotational actuation control 137 laterally (e.g., in a circumferential direction) by applying a lateral (e.g., circumferential) force to the scroll wheel with a finger, such as thumb, an index finger, or a middle finger.

In some embodiments, rotational actuation control 137 comprises a stud coupled to a chassis spindle 133 disposed within a housing of the cutting device. In some embodiments, chassis spindle 133 is rotatably coupled to housing 123 (e.g., by one or more of spindle pivot 134 or handle channel 126). In some embodiments, slot 138 (e.g., as shown in FIG. 1E) allows a practitioner to slide stud 138 to rotate cutting head 100 (e.g., by rotating chassis spindle 133 within housing 123). In some embodiments, rotational actuation control 137 comprises a button, switch, or other control that operates a rotation motor of the cutting device configured to rotate all or a portion of the components of the cutting device (e.g., cutting head 100 and, optionally, chassis spindle 133).

In some embodiments, an actuator control 135 and/or a rotational actuation control 137 of a cutting device comprises a lock. In some embodiments, a lock of is configured to secure actuator control 135 or rotational actuation control 137 (or portion thereof) in a given position. In some embodiments, a lock of a cutting device prevents an actuation control (e.g., a button, a switch, an actuator, or a lever of actuator control 135 or rotational actuation control 137) from being unintentionally activated. For example, in some embodiments, a lock of rotational actuation control 137 or actuator control 135 is configured to prevent rotational actuation control 137 or actuator control 135 from moving from a first position (e.g., an “off” position) to a second position (e.g., an “on” position), for example, to prevent cutting head 100 from rotating unintentionally (e.g., as a result of incidental contact to rotational actuation control 137). In some embodiments, a lock of a cutting device prevents an actuation control (e.g., a button, a switch, an actuator, or a lever) from being unintentionally deactivated.

In some embodiments, a lock comprises a grip coupled to an elongate member. In some embodiments, a lock is slidably coupled to a housing of a cutting device. In some embodiments, a lock or portion thereof (e.g., an elongate member of a lock) can be moved relative to a rotational actuation control 137. In some embodiments, the grip of a lock is contacted by a user to move the lock or a portion thereof into contact with or into an actuation path of an actuation control (e.g., rotational actuation control 137), for example, to physically block the actuation control from moving from a first position to a second position (e.g., from an “off” position to an “on” position or vice versa). In some embodiments, a lock comprises a mechanism for decoupling an actuation control (e.g., rotational actuation control 137) from a means of actuation (e.g., a motor, gear, actuator, shaft, or electrical circuit configured to actuate a cutting device or portion thereof, such as a cutting head).

In some embodiments, slot 138 comprises ridges or a lock for securing stud 137 in one or more positions along slot 138. In some embodiments, slot 138 comprises an arc about a longitudinal axis 140 of a cutting device of between 0 degrees and 360 degrees. In some embodiments, slot 138 comprises an arc about a longitudinal axis 140 of a cutting device of 5 degrees to 15 degrees, 5 degrees to 30 degrees, 5 degrees to 45 degrees, 5 degrees to 60 degrees, 5 degrees to 90 degrees, 5 degrees to 135 degrees, 5 degrees to 180 degrees, 5 degrees to 225 degrees, 5 degrees to 270 degrees, 5 degrees to 315 degrees, 5 degrees to 360 degrees, 15 degrees to 30 degrees, 15 degrees to 45 degrees, 15 degrees to 60 degrees, 15 degrees to 90 degrees, 15 degrees to 135 degrees, 15 degrees to 180 degrees, 15 degrees to 225 degrees, 15 degrees to 270 degrees, 15 degrees to 315 degrees, 15 degrees to 360 degrees, 30 degrees to 45 degrees, 30 degrees to 60 degrees, 30 degrees to 90 degrees, 30 degrees to 135 degrees, 30 degrees to 180 degrees, 30 degrees to 225 degrees, 30 degrees to 270 degrees, 30 degrees to 315 degrees, 30 degrees to 360 degrees, 45 degrees to 60 degrees, 45 degrees to 90 degrees, 45 degrees to 135 degrees, 45 degrees to 180 degrees, 45 degrees to 225 degrees, 45 degrees to 270 degrees, 45 degrees to 315 degrees, 45 degrees to 360 degrees, 60 degrees to 90 degrees, 60 degrees to 135 degrees, 60 degrees to 180 degrees, 60 degrees to 225 degrees, 60 degrees to 270 degrees, 60 degrees to 315 degrees, 60 degrees to 360 degrees, 90 degrees to 135 degrees, 90 degrees to 180 degrees, 90 degrees to 225 degrees, 90 degrees to 270 degrees, 90 degrees to 315 degrees, 90 degrees to 360 degrees, 135 degrees to 180 degrees, 135 degrees to 225 degrees, 135 degrees to 270 degrees, 135 degrees to 315 degrees, 135 degrees to 360 degrees, 180 degrees to 225 degrees, 180 degrees to 270 degrees, 180 degrees to 315 degrees, 180 degrees to 360 degrees, 225 degrees to 270 degrees, 225 degrees to 315 degrees, 225 degrees to 360 degrees, 270 degrees to 315 degrees, 270 degrees to 360 degrees, or 315 degrees to 360 degrees. In some embodiments, slot 138 comprises an arc about a longitudinal axis 140 of a cutting device of 5 degrees, 15 degrees, 30 degrees, 45 degrees, 60 degrees, 90 degrees, 135 degrees, 180 degrees, 225 degrees, 270 degrees, 315 degrees, or 360 degrees. In some embodiments, slot 138 comprises an arc about a longitudinal axis 140 of a cutting device of at least 5 degrees, 15 degrees, 30 degrees, 45 degrees, 60 degrees, 90 degrees, 135 degrees, 180 degrees, 225 degrees, 270 degrees, or 315 degrees. In some embodiments, slot 138 comprises an arc about a longitudinal axis 140 of a cutting device of at most 15 degrees, 30 degrees, 45 degrees, 60 degrees, 90 degrees, 135 degrees, 180 degrees, 225 degrees, 270 degrees, 315 degrees, or 360 degrees. In some embodiments, slot 138 comprises one or more ridges (e.g., to secure cutting head 100 in position at a given position) along its arc at a 0 degree position, a 5 degree position, a 15 degree position, a 30 degree position, a 45 degree position, a 60 degree position, a 90 degree position, a 135 degree position, a 180 degree position, a 225 degree position, a 270 degree position, a 315 degree position, a 360 degree position, or at a position between any two of these positions.

In some embodiments, rotational actuation control 137 and/or one or more of housing 123, spindle 133, or spindle pivot 134 comprises one or more physical features (e.g., notches, protrusions, or stops) that maintain the cutting head 100 in a first position relative to housing 123 and/or one or more other components of a cutting device, such as spindle 133 and/or rotational actuation control 137. In some embodiments, pressure applied to rotational actuation control (e.g., in a lateral or circumferential direction) can overcome the force of the one or more physical features configured to maintain the position of cutting head 100 so that cutting head can be rotated or rotated further along a circumferential arc 142 about longitudinal axis 140. Optionally, a second feature or plurality of features can be configured to maintain the cutting head 100 in a second position relative to housing 123 and/or one or more other components of the cutting device. For example, a rotational actuation control 137 comprising a scroll wheel comprises notches that allow a user to rotate cutting head 100 precisely and consistently from a first position to a second position around longitudinal axis 140, in some embodiments.

In some embodiments, rotational actuation control 137 comprises texturing 139 to improve control in the operation of rotational actuation control 137. For example, one or more surfaces of rotational actuation control 137 can comprise one or more features, such as indentations (e.g., as shown in FIG. 1F), grooves, patterned or unpatterned texturing (e.g., comprising vertical, horizontal, diagonal, circular, or cross-hatched surface features). In some embodiments, a surface of rotational actuation control 137 (e.g., a surface contacted by a user to operate rotational actuation control 137) is smooth. In some embodiments, all or a portion of rotational actuation control 137 comprises a material that offers improved control of rotational actuation control 137, such as a material that offers increased friction when in contact with skin or sterile glove materials (e.g., materials comprising natural rubber, latex, synthetic rubber, acrylonitrile butadiene rubber, butyl rubber, polyvinyl chloride, and/or neoprene).

A cutting device disclosed herein comprises 1, 2, 3, 4, 5, 6, 7, 8, 9, 10, or more than 10 rotational actuation controls 137 configured to operate an oscillator. In some embodiments, a cutting device comprises one or more rotational actuation controls 137 configured to rotate a cutting head 100. In some embodiments, a cutting device comprises one or more rotational actuation controls configured to stop the rotation of a cutting head 100. In some embodiments, a rotational actuation control 137 of a cutting device is configured to activate an oscillator based on a first set of one or more actuations (e.g., wherein a set of one or more actuations comprises a single actuation or a plurality of actuations of the actuation control) and to deactivate an oscillator based on a second set of one or more actuations (e.g., wherein a set of one or more actuations comprises a single actuation or a plurality of actuations of the actuation control).

In many embodiments, a switch, button, or touchpad of rotational actuation control 137 is configured to activate (e.g., turn on) at least one component of rotational actuation control 137 (e.g., to activate a motor or engage a gear of rotational actuation control 137). In some embodiments, a switch, button, or touchpad of rotational actuation control 137 is configured to deactivate (e.g., turn off) at least one component of rotational actuation control 137 (e.g., to deactivate a motor or disengage a gear of rotational actuation control 137). In some embodiments, a switch, button, or touchpad of rotational actuation control 137 is configured to activate and deactivate at least one component of rotational actuation control 137. In some embodiments, a switch, button, or touchpad comprises a first position and a second position, the first position corresponding to a first functionality of the switch, button, or touchpad (e.g., activation of rotational actuation control 137 or a component thereof) and the second position corresponding to a second functionality of the switch, button, or touchpad (e.g., deactivation of rotational actuation control 137 or a component thereof).

A switch, button, or touchpad of a cutting device disclosed herein (e.g., a switch, button, or touchpad of actuator control 135 and/or rotational actuation control 137) has 1, 2, 3, 4, 5, 6, 7, 8, 9, 10, or more than 10 positions. In some embodiments, each position of a switch, button, or touchpad corresponds to a different functionality of the switch, button, or touchpad. In some embodiments, placing a switch, button, or touchpad in a given position (e.g., by depressing or sliding a button, switch, or touchpad fully or partially, for example, to an intermediate position) causes a cutting head 100 to begin moving (e.g., along rotational arc 142, in a reciprocal motion, or vibrating), to stop moving in a given direction (e.g., along rotational arc 142, in a reciprocal motion, or vibrating), to change directions of motion (e.g., along rotational arc 142), or to change the speed at which cutting head 100 is moving.

In many cases, housing 123 comprises a compartment for internal components of a cutting device. One or more batteries 136 is housed within housing 123, in many embodiments. One or more batteries housed within cutting device 102 are used to power oscillator 128, in many embodiments. In some embodiments, housing 123 comprises an upper portion and a lower housing 124. In some embodiments, an upper portion of housing 123 and lower housing 124 are removably coupled to one another (e.g., to allow access to internal components for cleaning or maintenance of components, such as changing batteries). For example, one or more of an upper portion of housing 123 or lower housing 124 can comprise one or more features 125 (e.g., comprising lips, ridges, clips or other connectors) used to couple (e.g., snap) an upper portion of housing 123 and lower housing 124 together (e.g., as shown in FIG. 1D). In some embodiments, an upper portion of housing 123 and lower housing 124 comprise corresponding grooves that can be used to screw the upper and lower portions of housing 123 together. In some embodiments, all or a portion of housing 123 serves as a handle or grip of cutting device 102 for manual manipulation of cutting device 102. Housing 123 and/or lower housing section 124 comprises a plastic or metal material, in many embodiments. For example, the housing 123 (and/or lower housing 124) comprise an autoclavable plastic or metal material in some embodiments. In many cases, housing 123 and/or lower housing 124 comprise a stiff material to aid in efficiently transmitting force from a user's hand to a portion of cutting head 100 in contact with a biological tissue (e.g., an eye tissue of a patient). In many cases, a structural material of housing 123 and/or lower housing 124 (e.g., a metal or plastic material) is selected for its durability under prolonged exposure to vibrational forces. In some embodiments, housing 123 and/or lower housing 124 comprises a coating or layer of material for comfort or ergonomic benefit of a user. For example, a housing 123 and/or lower housing 124 comprises a soft plastic or composite material, such as a gel pad, to reduce vibrational forces on the user's hand. In some embodiments, a structural material, coating, or layer of material on a housing 123 or lower housing 124 is shaped to conform to a user's hand or portion thereof (e.g., for ease of control over the device and comfort for users).

### Cutting Heads

FIGS. 2A-2D show views of cutting heads 100. In some embodiments, all or a portion of cutting head 100 is flat (e.g., as shown in FIG. 2B and FIG. 2D). In some embodiments, cutting head 100 comprises one or more curved portions (e.g., as shown in FIGS. 2F-21). In some embodiments, cutting head 100 comprises a wire. In some embodiments, a cutting head 100 comprising one or more curved portions is formed from a wire stock. In some embodiments, a wire stock used to form cutting head 100 is comprises a cross-section that is a regular shape. For example, a cross-section of a cutting head (or portion thereof) or of a wire stock used to form cutting head 100 is substantially round, substantially elliptical, substantially oval-shaped, substantially circular, substantially semicircular, substantially square, substantially rectangular, or substantially trapezoidal, in some embodiments. It is contemplated that a cross-section of a wire stock or finished cutting head can have an irregular shape. In some embodiments, a wire stock or portion thereof is bent or otherwise formed into a cutting head having a handle 114 and at least one curved portion (e.g., curved first portion 108). In some embodiments, cutting head 100 comprises a wire having a curved (e.g., arced) first portion 108. In some embodiments, a curve of cutting head 100 comprises a circular arc. In some embodiments, a curve of cutting head 100 comprises an elliptical arc. In some embodiments, a curve of cutting head 100 comprises an ellipsoid arc. In some embodiments, a curve of cutting head 100 comprises an oval arc. In some embodiments, a curve of cutting head 100 comprises an ovoid arc. In some embodiments, a curve of cutting head 100 comprises a variable radius arc that is non-circular. In some embodiments, a curve of cutting head 100 comprises a non-straight path along its length from the proximal end to the tip of the cutting head.

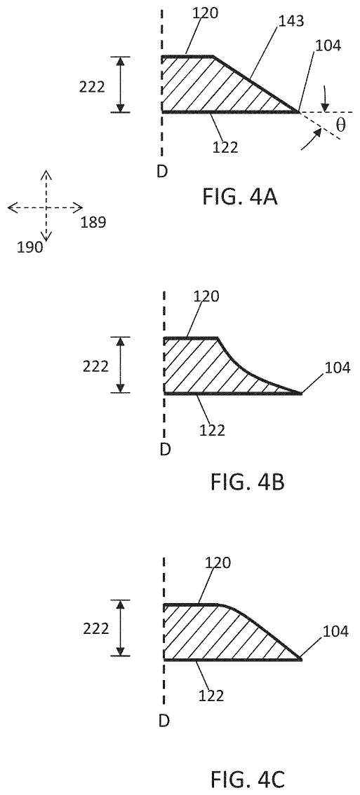

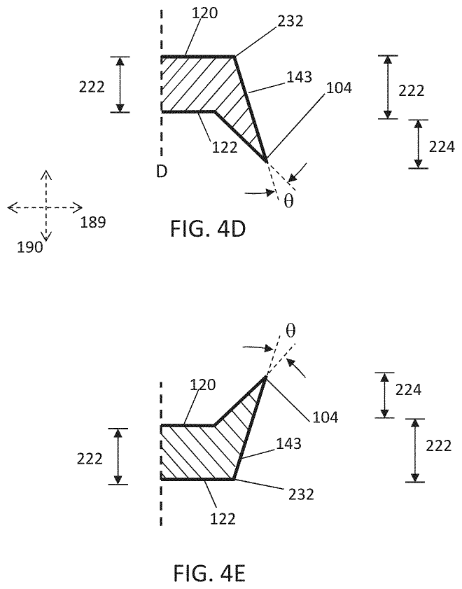

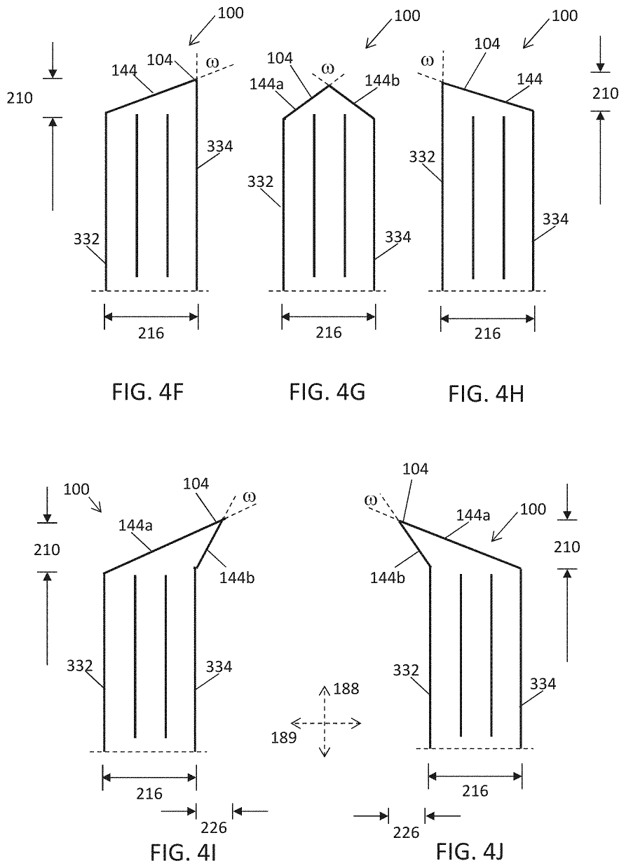

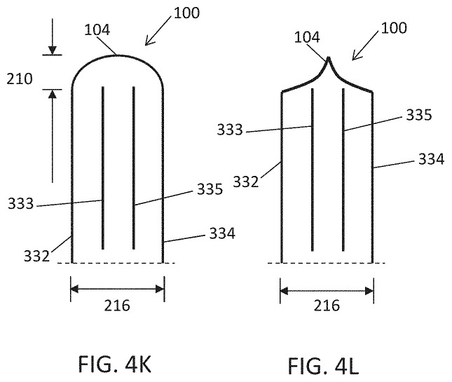

In many embodiments, wire stock is processed to include one or more features of a cutting head 100, as disclosed herein. In some embodiments, cutting head 100 comprises a pointed distal tip 104 (e.g., as shown in FIGS. 4F-4J and FIG. 4L). In some embodiments, distal tip 104 of cutting head 100 is needle-shaped. In some embodiments, a cutting head 100 comprises a rounded distal tip 104 (e.g., as shown in FIG. 2A, FIG. 2H, and FIG. 4K). It is also contemplated that a distal tip 104 can be blunt-tipped (e.g., not rounded or pointed), for example, as shown in FIG. 2C. The shape of the distal tip (e.g., pointed, rounded, or blunt-ended) can be selected based on the cutting procedure to be performed (e.g., a medical procedure, such as eye lens cutting, eye capsule cutting, or cutting of a histological tissue). An advantage of the cutting device systems disclosed herein is that cutting heads with different shapes, lengths, widths, and/or features can be exchanged between or during procedures, in some embodiments.

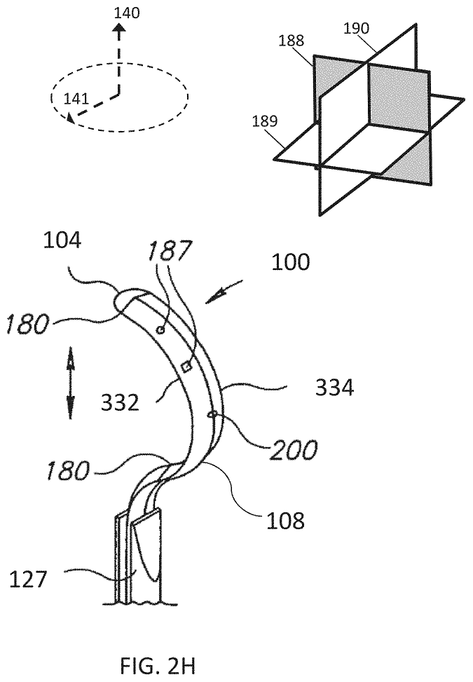

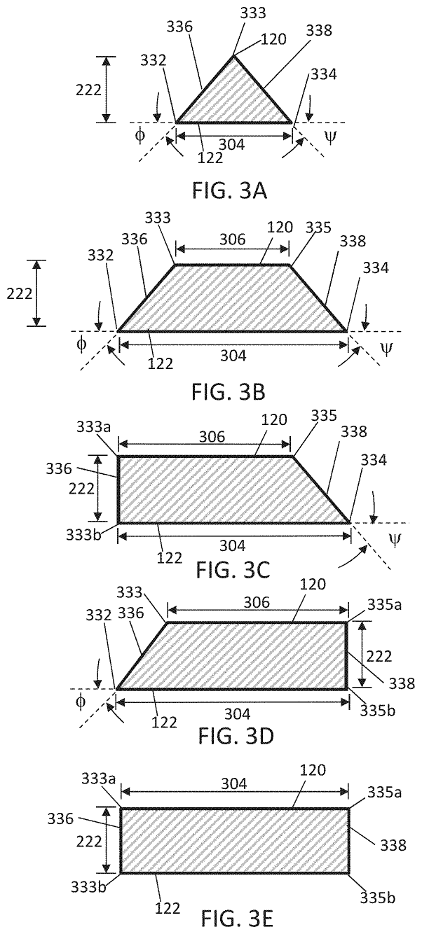

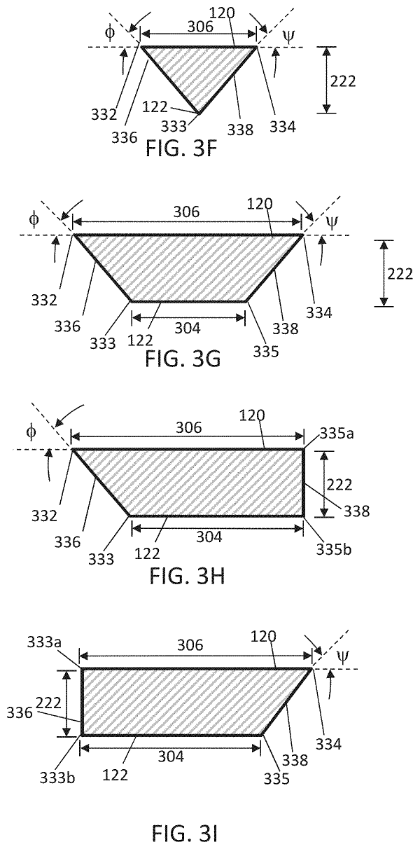

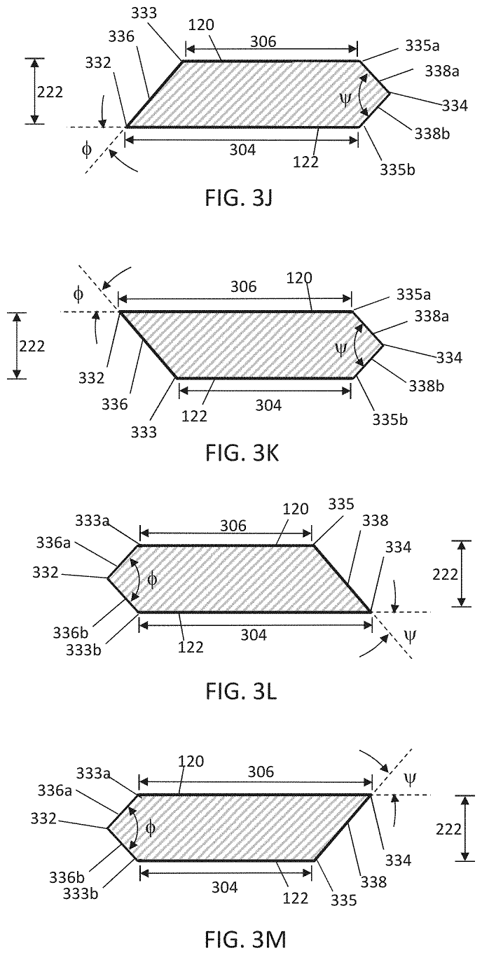

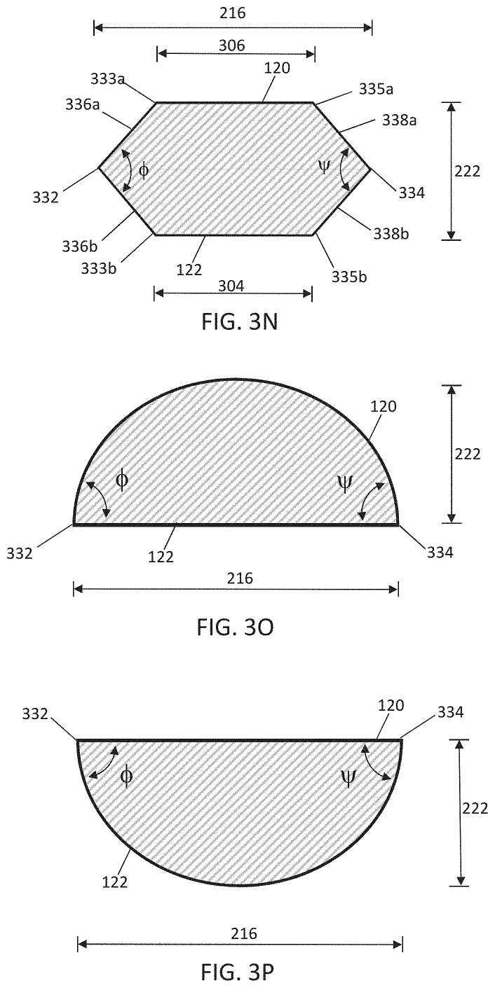

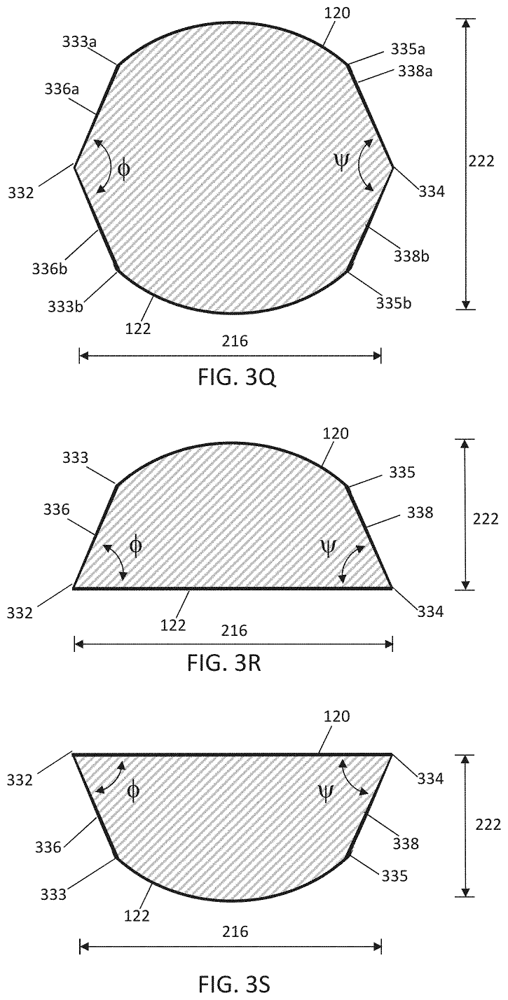

In many embodiments, a wire stock is cut or ground along all or a portion of its length (e.g., all or a portion of cutting head 100 indicated by length 210 and/or length 212 in FIG. 2C, which can include first portion 108 and/or distal tip 104) to form a sharp cutting edge (e.g., such as cutting or sharp edges 332 and 334, as shown in FIGS. 3A-3S). In some embodiments, a straight wire (e.g., a wire stock that has been processed to include one or more cutting edge, for example, by grinding or cutting the wire stock) is formed into a cutting head 100 comprising one or more curved portions prior to use. In some embodiments, a wire stock having a substantially rectangular or substantially square cross-section (e.g., having a cross-sectional shape shown in FIG. 3E) is ground or cut (e.g., along all or a portion of its longitudinal length, such as first portion 108 and/or distal tip 104) to form a cutting head comprising one or more portion(s) (e.g., all or a portion of cutting head 108 and/or all or a portion of distal tip 104) having the transverse cross-section shown in any one of FIGS. 3A-3D or FIGS. 3F-3N. In some embodiments, a wire stock having a substantially circular cross-sectional shape (e.g., having a cross-sectional diameter 216) is ground or cut (e.g., along all or a portion of its longitudinal length, such as first portion 108 and/or distal tip 104) to form a cutting head comprising one or more portion(s) (e.g., all or a portion of first portion 108 and/or all or a portion of distal tip 104) having a transverse cross-section shown in any one of FIGS. 30-3S. In some embodiments, a handle 114 of cutting head 100 has the cross-section of the raw wire stock. In some embodiments, a wire stock is cut or ground to form the cross-sectional shape of handle 114. In many cases, a cutting edge of cutting head 100 is formed prior to bending or forming a wire stock into cutting head 100, for example, to simplify the process of cutting or grinding the cutting edge. In some embodiments, a cross-section of cutting head 100 comprises one or more edges (e.g., cutting head corners 333, 333a, 333b, 335, 335a, and 335b, as shown in FIGS. 3A-3S) that are not sharpened (e.g., blunt or rounded edges, which can reduce the likelihood of unintended cuts to a biological tissue).

In some embodiments, a processed wire (e.g., a wire stock that has been cut or ground to form at least one cutting edge and/or distal tip geometry, as disclosed herein) from which a cutting head 100 is formed comprises a different shape and/or different dimensions at two or more points along its longitudinal length. In some embodiments, a cutting head 100 comprises a different shape and/or different dimensions at two or more points along its longitudinal length. A cutting head 100 comprises a beveled distal tip (e.g., for formation of a sharp edge at or around a distal tip), in many cases. As shown in the exemplary embodiment shown in FIG. 2B, a bevel 202 of a cutting head 100 can meet a medial surface 120 or a lateral surface 122 of distal tip 104 at a shoulder 220. In some embodiments, a distal tip 104 of cutting head 100 has a length 210. In some embodiments, a distal tip length 210 is 0.1 mm to 5 mm. In some embodiments, a distal tip length 210 is 0.1 mm to 0.5 mm, 0.1 mm to 1 mm, 0.1 mm to 2 mm, 0.1 mm to 3 mm, 0.1 mm to 4 mm, 0.1 mm to 5 mm, 0.5 mm to 1 mm, 0.5 mm to 2 mm, 0.5 mm to 3 mm, 0.5 mm to 4 mm, 0.5 mm to 5 mm, 1 mm to 2 mm, 1 mm to 3 mm, 1 mm to 4 mm, 1 mm to 5 mm, 2 mm to 3 mm, 2 mm to 4 mm, 2 mm to 5 mm, 3 mm to 4 mm, 3 mm to 5 mm, or 4 mm to 5 mm. In some embodiments, a distal tip length 210 is 0.1 mm, 0.5 mm, 1 mm, 2 mm, 3 mm, 4 mm, or 5 mm. In some embodiments, a distal tip length 210 is at least 0.1 mm, 0.5 mm, 1 mm, 2 mm, 3 mm, or 4 mm. In some embodiments, a distal tip length 210 is at most 0.5 mm, 1 mm, 2 mm, 3 mm, 4 mm, or 5 mm. In many embodiments, cutting head 100 comprises a portion having one or more sharp edges. A portion of cutting head 100 having one or more sharp edge has a length 212, in many embodiments. A portion of cutting head 100 having no sharp edges can have a length 214 and may comprise all or a portion of handle 114. In some embodiments, length 214 is at least 1 mm, at least 2 mm, at least 3 mm, at least 4 mm, at least 5 mm, at least 6 mm, at least 7 mm, at least 8 mm at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, more than 20 mm, from 1 mm to 20 mm, from 3 mm to 17 mm, from 5 mm to 15 mm, from 7 mm to 13 mm, or from 9 mm to 11 mm. A cutting head has a width 216, in many embodiments. A cutting head has a thickness 222, in many embodiments. In some embodiments, the thickness 222 is constant or substantially constant from a proximal end of the cutting head 100 to a distal tip 104 of cutting head 100. In some embodiments, the thickness 222 can vary over a portion of the length of cutting head 100. For example, cutting head 100 can comprise a shoulder 220 between a portion of the cutting head having a full thickness and a portion of the cutting head 100 where the thickness of the cutting head is less than full thickness. In some embodiments, a cutting head 100 has a thickness of at least 0.1 mm, at least 0.5 mm, at least 1 mm, at least 1.5 mm, at least 2 mm, at least 3 mm, at least 4 mm, at least 5 mm, less than 0.1 mm, from 0.1 mm to 5 mm, from 0.1 mm to 1 mm, from 0.25 mm to 0.75 mm, or from 0.4 mm to 0.6 mm. In some embodiments, the thickness 222 (e.g., at a full thickness of cutting head 100) is 0.5 mm.

In some embodiments, (e.g., as exemplified in FIGS. 2E, 2F, 2G, 4D, and 4E), a distal tip 104 is angled or bent (e.g., in a sagittal plane 188 of cutting head) at bend 232 relative to an adjacent portion of cutting head 100 (e.g., an adjacent curved portion of cutting head 100). In some embodiments, bend 232 comprises an angle of 90 degrees. In some embodiments, bend 232 comprises an angle between 0 degrees and 180 degrees, from 1 degree to 90 degrees, from 1 degree to 30 degrees, from 30 degrees to 45 degrees, from 1 degree to 45 degrees, from 45 degrees to 90 degrees, from 45 degrees to 60 degrees, from 30 degrees to 60 degrees, from 90 degrees to less than 180 degrees, from 90 degrees to 135 degrees, or from 135 degrees to less than 180 degrees. In some embodiments, bend 232 comprises an angle of from 45 degrees to 135 degrees. In some embodiments, a deflection distance 224 of a distal tip past a medial surface 120 or lateral surface 122 of the cutting head is about 1% to about 500% of the thickness 222 of the cutting head wire. In some embodiments, a deflection distance 224 of a distal tip past a medial surface 120 or lateral surface 122 of the cutting head is about 1% to about 10%, about 1% to about 50%, about 1% to about 100%, about 1% to about 200%, about 1% to about 300%, about 1% to about 400%, about 1% to about 500%, about 10% to about 50%, about 10% to about 100%, about 10% to about 200%, about 10% to about 300%, about 10% to about 400%, about 10% to about 500%, about 50% to about 100%, about 50% to about 200%, about 50% to about 300%, about 50% to about 400%, about 50% to about 500%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, or about 400% to about 500% of the thickness 222 of the cutting head wire. In some embodiments, a deflection distance 224 of a distal tip past a medial surface 120 or lateral surface 122 of the cutting head is about 1%, about 10%, about 50%, about 100%, about 200%, about 300%, about 400%, or about 500% of the thickness 222 of the cutting head wire. In some embodiments, a deflection distance 224 of a distal tip past a medial surface 120 or lateral surface 122 of the cutting head is at least about 1%, about 10%, about 50%, about 100%, about 200%, about 300%, or about 400% of the thickness 222 of the cutting head wire. In some embodiments, a deflection distance 224 of a distal tip past a medial surface 120 or lateral surface 122 of the cutting head is at most about 10%, about 50%, about 100%, about 200%, about 300%, about 400%, or about 500% of the thickness 222 of the cutting head wire. Cutting heads that include a bend 232 at distal tip 104 can offer additional functionality, for example, in making incisions and cuts, such as cutting a capsule during an eye procedure. In some embodiments, distal tip 104 of cutting head 100 is not angled relative to the curve of a portion 108 of the cutting head 100 (e.g., as shown in FIG. 2H).

FIG. 2H is an enlarged perspective view of cutting head 100, in accordance with an exemplary embodiment of the invention. Cutting head 100 optionally has a semicircular shape for cutting a circular or semi-circular incision in the lens capsule. Cutting head 100 optionally has sharp edges 332 and 334 on both planar sides of the cutting head, such that cutting can be performed on either planar side of the cutting head. In some embodiments,

In many cases, cutting head 100 or a portion thereof is symmetrical with respect to a sagittal (e.g., a midsagittal) plane 188. In some embodiments, a distal tip is symmetrical with respect to a sagittal plane 188. In some embodiments, cutting head 100 is asymmetrical with respect to sagittal plane 188. In some embodiments, a transverse cross-section of a first portion of cutting head 100 is symmetrical (e.g., as shown in FIG. 4G, FIG. 4K, and FIG. 4L). In some embodiments, a distal tip is asymmetrical with respect to sagittal plane 188 (e.g., as shown in FIG. 4F, FIG. 4H, FIG. 4I, FIG. 4J). FIG. 2H also shows the planes referred to herein—including the sagittal plane 188, coronal plane 190 and transverse plane 189. As used herein the lateral and medial aspects of the device (surface, side or other lateral aspect are noted with respect to the coronal plane 190 as shown in FIG. 2H. If the device is flipped 180 degrees along its longitudinal axis, the lateral and medial aspects of the device are flipped also relative to the coronal plane 190. FIG. 2H also depicts the transverse plane 189 used herein.

Sharp edges 332 and 334 optionally extend over substantially the entire extent of cutting head 100. In some embodiments of the invention, sharp edges 332 and 334 do not extend over a distal tip 104 of cutting head 100, so that distal tip 104 does not cause inadvertent cutting when inserted into the patient's eye. Alternatively, sharp edges 332 and/or 334 extend over the entire distal tip 104 of cutting head 100. For example, distal tip edge 144, 144a, and/or 144b (e.g., as shown in FIGS. 4F-4J, alternatively called a tip edge or tip edges herein) are sharp edges, in some embodiments. In some embodiments, a sharp edge is formed at distal tip edge 144, 144a and/or 144b by cutting or grinding a medial surface or lateral surface (or both) of wire stock to form a distal tip beveled edge 143 in the sagittal cross-section of distal tip 104 (e.g., as shown in FIGS. 4A, 4D, and 4E). In some embodiments, all or a portion of a first portion 108, a second portion 109, or any subsequent portions of cutting head 100 can comprise one or more sharp edges. In some embodiments of the invention, sharp edges 332 and 334 do not extend to a connection point of cutting head 100 with handle 114, so as to limit the chance of inadvertent undesired cutting. In an exemplary embodiment of the invention, sharp edges 332 and 334 extend over about 145°-165° of a curved portion of cutting head 100. Alternatively, sharp edges 332 and 334 extend over at least 180° of a curved portion of cutting head 100, so that a complete circle cut can be made when a circular cut of a maximal radius of cutting head 100 is desired.

In some embodiments of the invention, sharp edges 332 and 334 have a same extent (i.e. same lengths along the cutting head 100). Alternatively, sharp edges 332 and 334 have different extents, allowing formation of unsymmetrical cuts, when required.

The size of cutting head 100 is optionally a compromise between a large size for achieving large cutting edges and a small size which is easier to manipulate in the anterior chamber. In some embodiments of the invention, cutting head 100 extends over about half a circle, e.g., about 175°-185°, such that a distal tip 104 of cutting head 100 is substantially on the longitudinal axis of the cutting device.

### Cutting Head Shape

Cutting head 100 can have a shape that defines an incision having a substantial area. Optionally, cutting head 100 can be defined as a portion of a substantially perfect circle. Alternatively, in order to achieve a non-circular cut, a semi-elliptical (e.g., half an ellipse) shaped, a partial ellipsis shaped, an oval shaped, a partial oval shaped, a variable radius curved shaped, or any non-straight path shaped cutting head can be used. Further alternatively, a triangular or rectangular shape, or any other shape can be used for cutting head 100. In some embodiments of the invention, cutting head 100 can be formed from a relatively soft material or structure, which can allow a physician to adjust the shape of cutting head 100. Optionally, cutting head 100 can be relatively rigid in the cutting direction, while being relatively flexible in a direction allowing adjustment of the planar shape of the cutting head.

In some embodiments of the invention, rather than having an open shape, cutting head 100 can have a closed shape, such as a full circle or elliptic shape. Optionally, the cutting head in accordance with this embodiment can have a flexible shape which can be condensed for insertion into the eye. After insertion, the cutting head can be expanded to its cutting shape, for example as described in above mentioned U.S. Pat. No. 5,728,117. After the expanding of the cutting head, the cutting head can be actuated (e.g., oscillated or vibrated) to perform the cutting.

A cutting head 100 can comprise various cross-sections, including one or more of those shown in FIGS. 3A-3S. Cross-sections A, B, and C of cutting head 100, as indicated in FIGS. 2A, 2C, and 2E can comprise any shape, including triangles (e.g., as shown in FIG. 3A and FIG. 3F), trapezoids (e.g., as shown in FIGS. 3B-3D and FIG. 3G-3I), rectangles (e.g., as shown in FIG. 3E), squares, pentagons (e.g., as shown in FIGS. 3J-3M), hexagons (e.g., as shown in FIG. 3N), octagons, or polygons having other numbers of sides, ovals, circles, semicircles (e.g., as shown in FIG. 3O and FIG. 3P) or irregular shapes, such as star shapes, beveled circles (e.g., as shown in FIG. 3Q), and beveled semicircles (e.g., as shown in FIG. 3R and FIG. 3S). Examples of cross-sectional shapes are provided herein as viewed in transverse cross-section along transverse plane 189 and at farthest point 200 at which point the cross section is substantially parallel to the transverse plane 189. Such cross-sectional shapes of cutting head 100 are shown in FIGS. 3A-3S. Although the cross-sectional shapes of FIGS. 3A-3S are shown at point 200, the first portion may have the same cross-sectional shape at every point along its length from the distal tip until the bend, or for any sub-portion of the first portion. Of particular note are shapes that can comprise sharp edges, as these cross-sectional shapes may be useful for cutting edges of cutting head 100 (e.g., along one or more curved portion of cutting head 100). For example, edges 332 and/or 334 of FIGS. 3A-3D and 3F-3S are sharp edges in some embodiments. In some embodiments, a trapezoidal cross-section of a cutting head 100 comprises two sharp edges (e.g., as shown in FIG. 3B). In some embodiments, a cutting head 100 comprising two sharp edges (e.g., a trapezoidal cross-section or triangular cross-section with two sharp edges) allows for easy manufacturing of a two-sided cutting head 100. In some embodiments, a cutting head 100 comprising two sharp edges (e.g., a trapezoidal cross-section, a pentagonal cross-section, a semicircular cross-section, a beveled semicircular cross-section, a beveled circular cross-section, or triangular cross-section comprising two sharp edges) is useful when a cutting head 100 is rotationally actuated, as disclosed herein.

In some embodiments, an edge (e.g., edge 332 or edge 334) is substantially aligned with a surface (e.g., a lateral surface 122 or a medial surface 120 of cutting head 100) when evaluated in a transverse cross-section of the cutting head 100. In some embodiments, an edge (e.g., edge 332 or edge 334) is substantially aligned with a surface when the edge is a distance of less than 2 percent to less than 25 percent of a thickness of cutting head 100 from the surface (e.g., measured perpendicularly in the transverse cross-section from the surface). In some embodiments, an edge (e.g., edge 332 or edge 334) is substantially aligned with a surface when the edge is a distance of less than 2 percent, less than 5 percent, less than 10 percent, less than 15 percent, less than 20 percent, or less than 25 percent of a thickness of cutting head 100 from the surface (e.g., measured perpendicularly in the transverse cross-section from the surface). In some embodiments, an edge (e.g., edge 332 or edge 334) is substantially aligned with a surface when the edge is a distance of less than at most 5 percent, at most 10 percent, at most 15 percent, at most 20 percent, or at most 25 percent of a thickness of cutting head 100 from the surface (e.g., measured perpendicularly in the transverse cross-section from the surface).

In some embodiments, a sharp edge (e.g., edge 332 or edge 334) is not substantially aligned with a surface (e.g., lateral surface 122 or medial surface 120) when evaluated in a transverse cross-section of the cutting head 100. In some embodiments, an edge (e.g., edge 332 or edge 334) is not substantially aligned with a surface when the edge is a distance of greater than 25 percent to greater than 50 percent of a thickness of cutting head 100 from the surface (e.g., measured perpendicularly in the transverse cross-section from the surface). In some embodiments, an edge (e.g., edge 332 or edge 334) is not substantially aligned with a surface when the edge is a distance of greater than 25 percent, greater than 30 percent, greater than 35 percent, greater than 40 percent, greater than 45 percent, or greater than 50 percent of a thickness of cutting head 100 from the surface (e.g., measured perpendicularly in the transverse cross-section from the surface). In some embodiments, an edge (e.g., edge 332 or edge 334) is not substantially aligned with a surface when the edge is a distance of at least 25 percent, at least 30 percent, at least 35 percent, at least 40 percent, at least 45 percent, or at least 50 percent of a thickness of cutting head 100 from the surface (e.g., measured perpendicularly in the transverse cross-section from the surface).

In some embodiments, a cross-sectional shape with no sharp edges (e.g., cross-sectional shapes comprising rectangular, square, oval shaped, and circular cross-sections, for example, as shown in FIG. 3E) is useful in the formation of a handle 114 of a cutting head 100.

In many cases, surface 336 (e.g., connecting surface 336, 336a, 336b, or a portion thereof) of cutting head 100 is adjacent (e.g., circumferentially connected and circumferentially adjacent) to medial surface 120 and lateral surface 122. For example, when cutting head 100 or a portion thereof (e.g., first portion 108) is viewed in transverse cross-section, surface 336 (e.g., connecting surface 336, 336a, 336b, or a portion thereof) is circumferentially adjacent to medial surface 120, in some embodiments. In some embodiments, surface 336 (e.g., connecting surface 336, 336a, 336b, or a portion thereof) is circumferentially adjacent to lateral surface 122, when cutting head 100 or a portion thereof (e.g., first portion 108) is viewed in transverse cross-section. In some embodiments, surface 336 (e.g., connecting surface 336, 336a, 336b, or a portion thereof) is circumferentially adjacent to medial surface 120 and lateral surface 122, when cutting head 100 or a portion thereof (e.g., first portion 108) is viewed in transverse cross-section. In many cases, surface 338 (e.g., connecting surface 338, 338a, 338b, or a portion thereof) of cutting head 100 is adjacent (e.g., circumferentially connected and circumferentially adjacent) to medial surface 120 and lateral surface 122. For example, when cutting head 100 or a portion thereof (e.g., first portion 108) is viewed in transverse cross-section, surface 338 (e.g., connecting surface 338, 338a, 338b, or a portion thereof) is circumferentially adjacent to medial surface 120, in some embodiments. In some embodiments, surface 338 (e.g., connecting surface 338, 338a, 338b, or a portion thereof) is circumferentially adjacent to lateral surface 122, when cutting head 100 or a portion thereof (e.g., first portion 108) is viewed in transverse cross-section. In some embodiments, surface 338 (e.g., connecting surface 338, 338a, 338b, or a portion thereof) is circumferentially adjacent to medial surface 120 and lateral surface 122, when cutting head 100 or a portion thereof (e.g., first portion 108) is viewed in transverse cross-section. In many cases, surface 336 (e.g., surface 336, 336a, 336b, or a portion thereof) is not adjacent (e.g., not circumferentially adjacent) to surface 338 (e.g., surface 338, 338a, 338b, or a portion thereof). In some embodiments, surface 336 (e.g., surface 336, 336a, 336b, or a portion thereof) is perpendicular to medial surface 120. In some embodiments, surface 336 (e.g., surface 336, 336a, 336b, or a portion thereof) is perpendicular to lateral surface 122. In some embodiments, surface 336 (or a portion thereof) is perpendicular to both medial surface 120 and lateral surface 122. In some embodiments, surface 338 (e.g., surface 338, 338a, 338b, or a portion thereof) is perpendicular to medial surface 120. In some embodiments, surface 338 (e.g., surface 338, 338a, 338b, or a portion thereof) is perpendicular to lateral surface 122.

In some embodiments, surface 336 is adjacent (e.g., circumferentially connected and circumferentially adjacent) to surface 338 (e.g., as shown in FIG. 3A and FIG. 3F, wherein medial surface 120 or lateral surface 122 comprises an edge). In many cases, medial surface 120 is not adjacent (e.g., not circumferentially adjacent) to lateral surface 122 (e.g., as shown in FIGS. 3B-3E, 3G-3N, and 3Q-3S). In some embodiments, medial surface 120 is adjacent (e.g., circumferentially connected and circumferentially adjacent) to lateral surface 122 (e.g., as shown in FIGS. 30 and 3P). In many cases, medial surface 120 or a portion thereof is substantially flat. In some embodiments, medial surface 120 or a portion thereof is not substantially flat. In many cases, lateral surface 122 or a portion thereof is substantially flat. In some embodiments, lateral surface 122 or a portion thereof is not substantially flat.

In some embodiments, surface 336 (or surface 336a) meets medial surface 120 at edge 333 (or edge 333a). In some embodiments, surface 336 (or surface 336a) meets lateral surface 122 at edge 333 (or edge 333b). In some embodiments, surface 338 (or surface 338a) meets medial surface 120 at edge 335 (or edge 335a). In some embodiments, surface 336 (or surface 336a) meets lateral surface 120 at edge 335 (or edge 335b). In some embodiments, surface 336a meets surface 336b at edge 332. In some embodiments, surface 336 or surface 336a meets medial surface 120 at edge 332. In some embodiments, surface 336 or surface 336b meets lateral surface 122 at edge 332. In some embodiments, surface 338a meets surface 338b at edge 334. In some embodiments, surface 338 or surface 338a meets medial surface 120 at edge 334. In some embodiments, surface 338 or surface 338b meets lateral surface 122 at edge 334. In some embodiments, edge 332 is a sharp edge. In some embodiments, edge 334 is a sharp edge. In some embodiments, edges 333, 333a, 333b, 335, 335a, and/or 335b are not sharp edges. For example, in some embodiments, edges 333, 333a, 333b, 335, 335a, and/or 335b are rounded or blunt edges.

In some embodiments, a beveled surface (e.g., surface 336, surface 336a, surface 336b, surface 338, surface 338a, and/or surface 338b) comprises a sharp edge of cutting head 100 (e.g., edge 332 and/or edge 334). In some embodiments, a first beveled surface 336 is at an angle of phi (□□ relative to a medial surface 120 or a lateral surface 122 of cutting head 100. In some embodiments, a first beveled surface 336 is directly connected to a medial surface 120 at a first edge (e.g., edge 333), is directly connected to a lateral surface 122 at a second edge (e.g., edge 332), and is at an angle of phi (□□ relative to the medial surface 120. In some embodiments, a first beveled surface 336 is directly connected to a medial surface 120 at a first edge (e.g., edge 333), is directly connected to a lateral surface 122 at a second edge (e.g., edge 332), and is at an angle of phi (□□ relative to the lateral surface 122 of cutting head 100. In some embodiments, a second beveled surface 338 is at an angle psi (□□ relative to a medial surface 120 or a lateral surface 122 of cutting head 100. In some embodiments, a second beveled surface 338 is directly connected to a medial surface 120 at a first edge (e.g., edge 334), is directly connected to a lateral surface at a second edge (e.g., edge 335), and is at an angle psi (□□ relative to the medial surface 120. In some embodiments, a second beveled surface 338 is directly connected to a medial surface 120 at a first edge (e.g., edge 334), is directly connected to a lateral surface at a second edge (e.g., edge 335), and is at an angle psi (□□ relative to the lateral surface 122 of cutting head 100. In some embodiments, a cutting head 100 comprises both a first beveled surface at an angle of □ to a medial surface 120 or a lateral surface 122 of cutting head 100 and a second beveled surface 338 at an angle of □ to a medial surface 120 or a lateral surface 122 of cutting head 100. In some embodiments, a cutting head 100 comprises both a first beveled surface (e.g., surface 336) connected to a medial surface 120 at a first edge (e.g., edge 332) and to a lateral surface 122 at a second edge (e.g., edge 333) and is at an angle of □ to the medial surface 120 and a second beveled surface 338 is connected to medial surface 120 or lateral surface 122 of cutting head 100 at an angle of □. In some embodiments, a cutting head 100 comprises both a first beveled surface (e.g., surface 336) connected to a medial surface 120 at a first edge (e.g., edge 332) and to lateral surface 122 at a second edge (e.g., edge 333) and is at an angle of □ to the lateral surface 122 of cutting head 100 and a second beveled surface 338 is connected to medial surface 120 or lateral surface 122 of cutting head 100 at an angle of □. In some embodiments, angle □ is from 0 degrees to 90 degrees, from 0 degrees to 30 degrees, from 30 degrees to 45 degrees, from 0 degrees to 45 degrees, from 45 degrees to 90 degrees, from 45 degrees to 60 degrees, from 30 degrees to 60 degrees, from 90 degrees to less than 180 degrees, from 90 degrees to 135 degrees, or from 135 degrees to less than 180 degrees. In some embodiments, angle □ is from 0 degrees to 90 degrees, from 0 degrees to 30 degrees, from 30 degrees to 45 degrees, from 0 degrees to 45 degrees, from 45 degrees to 90 degrees, from 45 degrees to 60 degrees, from 30 degrees to 60 degrees, from 90 degrees to less than 180 degrees, from 90 degrees to 135 degrees, or from 135 degrees to less than 180 degrees.

In some embodiments, surface 338 (or a portion thereof) is perpendicular to both medial surface 120 and lateral surface 122. In some embodiments, surface 336 (e.g., surface 336, 336a, or a portion thereof) forms an angle □ with medial surface 120. In some embodiments, surface 336 (e.g., surface 336, 336b, or a portion thereof) forms an angle □ with lateral surface 122. In some embodiments, surface 336a forms an angle □ with surface 336b. When surface 336, 336a, or 336b meets medial surface 120 or lateral surface 122 at edge 332, angle □ is from 1 degree to 90 degrees. When surface 336, 336a, or 336b meets medial surface 120 or lateral surface 122 at edge 332, angle □ is from 1 degree to 30 degrees, 1 degree to 45 degrees, 1 degree to 60 degrees, 1 degree to 90 degrees, 30 degrees to 45 degrees, 30 degrees to 60 degrees, 30 degrees to 90 degrees, 45 degrees to 60 degrees, 45 degrees to 90 degrees, or 60 degrees to 90 degrees. When surface 336, 336a, or 336b meets medial surface 120 or lateral surface 122 at edge 332, angle □ is from 1 degree, 30 degrees, 45 degrees, 60 degrees, or 90 degrees. When surface 336, 336a, or 336b meets medial surface 120 or lateral surface 122 at edge 332, angle □ is from at least 1 degree, 30 degrees, 45 degrees, or 60 degrees. When surface 336, 336a, or 336b meets medial surface 120 or lateral surface 122 at edge 332, angle □ is from at most 30 degrees, 45 degrees, 60 degrees, or 90 degrees. When surface 336a meets surface 336b at edge 332, angle □ is from 91 degrees to 179 degrees. When surface 336a meets surface 336b at edge 332, angle □ is from 91 degrees to 120 degrees, 91 degrees to 135 degrees, 91 degrees to 150 degrees, 91 degrees to 179 degrees, 120 degrees to 135 degrees, 120 degrees to 150 degrees, 120 degrees to 179 degrees, 135 degrees to 150 degrees, 135 degrees to 179 degrees, or 150 degrees to 179 degrees. When surface 336a meets surface 336b at edge 332, angle □ is from 91 degrees, 120 degrees, 135 degrees, 150 degrees, or 179 degrees. When surface 336a meets surface 336b at edge 332, angle □ is from at least 91 degrees, 120 degrees, 135 degrees, or 150 degrees. When surface 336a meets surface 336b at edge 332, angle □ is from at most 120 degrees, 135 degrees, 150 degrees, or 179 degrees.

In some embodiments, surface 338 (e.g., surface 338, 338a, or a portion thereof) forms an angle □ with medial surface 120. In some embodiments, surface 338 (e.g., surface 338, 338b, or a portion thereof) forms an angle □ with lateral surface 122. In some embodiments, surface 338a forms an angle □ with surface 338b. When surface 338, 338a, or 338b meets medial surface 120 or lateral surface 122 at edge 334, angle □ is from 1 degree to 90 degrees. When surface 338, 338a, or 338b meets medial surface 120 or lateral surface 122 at edge 334, angle □ is from 1 degree to 30 degrees, 1 degree to 45 degrees, 1 degree to 60 degrees, 1 degree to 90 degrees, 30 degrees to 45 degrees, 30 degrees to 60 degrees, 30 degrees to 90 degrees, 45 degrees to 60 degrees, 45 degrees to 90 degrees, or 60 degrees to 90 degrees. When surface 338, 338a, or 338b meets medial surface 120 or lateral surface 122 at edge 334, angle □ is from 1 degree, 30 degrees, 45 degrees, 60 degrees, or 90 degrees. When surface 338, 338a, or 338b meets medial surface 120 or lateral surface 122 at edge 334, angle □ is from at least 1 degree, 30 degrees, 45 degrees, or 60 degrees. When surface 338, 338a, or 338b meets medial surface 120 or lateral surface 122 at edge 334, angle □ is from at most 30 degrees, 45 degrees, 60 degrees, or 90 degrees. When surface 338a meets surface 338b at edge 334, angle □ is from 91 degrees to 179 degrees. When surface 338a meets surface 338b at edge 334, angle □ is from 91 degrees to 120 degrees, 91 degrees to 135 degrees, 91 degrees to 150 degrees, 91 degrees to 179 degrees, 120 degrees to 135 degrees, 120 degrees to 150 degrees, 120 degrees to 179 degrees, 135 degrees to 150 degrees, 135 degrees to 179 degrees, or 150 degrees to 179 degrees. When surface 338a meets surface 338b at edge 334, angle □ is from 91 degrees, 120 degrees, 135 degrees, 150 degrees, or 179 degrees. When surface 338a meets surface 338b at edge 334, angle □ is from at least 91 degrees, 120 degrees, 135 degrees, or 150 degrees. When surface 338a meets surface 338b at edge 334, angle □ is from at most 120 degrees, 135 degrees, 150 degrees, or 179 degrees.

In some embodiments, a medial surface 120 of cutting head 100 meets a beveled surface (e.g., surface 336 or surface 338) at an edge (e.g., edge 334 or edge 336) of cutting head 100. In some embodiments, an edge of cutting head 100 at which medial surface 120 meets a beveled surface of cutting head 100 comprises a sharp edge. In some embodiments, a lateral surface 122 of cutting head 100 meets a beveled surface (e.g., surface 336 or surface 338) at an edge (e.g., edge 334 or edge 336) of cutting head 100. In some embodiments, an edge of cutting head 100 at which lateral surface 122 meets a beveled surface of cutting head 100 comprises a sharp edge.

Some embodiments of cutting head 100 (or a portion of cutting head 100) comprise one or more of various sagittal cross-sections, such as those shown in FIGS. 4A-4E. When viewed in sagittal cross-section, some embodiments of distal tip 104 of cutting head 100 comprise a bevel comprising bevel surface 143 at an angle theta (□, e.g., as shown in FIG. 4A) to a medial surface 120 or a lateral surface 122 of cutting head 100, a concave sagittal cross-section (e.g., as shown in FIG. 4B), or a convex sagittal cross-section (e.g., as shown in FIG. 4C). Each type of sagittal cross-section for distal tip 104 of cutting head 100 can be fashioned to comprise a pointed tip or sharp edge. In some embodiments, angle theta is an angle of between 0 degrees and 90 degrees, from 1 degrees to 75 degrees, from 15 degrees to 85 degrees, from 20 degrees to 70 degrees, from 10 degrees to 45 degrees, from 30 degrees to 60 degrees, from 25 degrees to 65 degrees, from 15 degrees to 90 degrees, or from 15 degrees to 75 degrees, from 0 degrees to 90 degrees, from 0 degrees to 30 degrees, from 30 degrees to 45 degrees, from 0 degrees to 45 degrees, from 45 degrees to 90 degrees, from 45 degrees to 60 degrees, from 30 degrees to 60 degrees, from 90 degrees to less than 180 degrees, from 90 degrees to 135 degrees, or from 135 degrees to less than 180 degrees relative to a medial surface 120 or a lateral surface of cutting edge 100.

In some embodiments, distal tip 104 comprises a pointed tip. In some embodiments, distal tip 104 comprises a “pencil point” tip. In some embodiments, a “pencil point” tip at distal tip 104 comprises two or more distal tip edges 144 (alternatively called tip edges of the distal tip) angled within a coronal section of distal tip 104 (e.g., at an angle □□ as shown in FIG. 4G). In some embodiments, one or more distal tip edges 144 of distal tip 104 comprises a constant angle when viewed in-plane with distal tip 104 (e.g., as shown in FIG. 4F and FIG. 4H). In some embodiments, one or more distal tip edges 144 of distal tip 104 can be curved within the plane of distal tip 104 (e.g., as shown in FIG. 4K and FIG. 4L). A cutting head 100 comprising a pointed distal tip 104 can be useful for performing incisions (e.g., during an eye procedure, such as cataract surgery) other than those made using other edges of the cutting head 100 (e.g., sharp edges of a curved portion 108). For example, a distal tip 104 comprising a pointed tip (e.g., as shown in FIG. 4F-FIG. 4J and FIG. 4L) can be used to make an incision in the capsule of an eye (e.g., prior to insertion of cutting head 100 into the capsule during an eye procedure, such as cataract surgery). In some embodiments, distal tip 104 is needle-shaped. In some embodiments, distal tip 104 comprises an edge (e.g., distal tip edge 144). In some embodiments, distal tip 104 comprises a plurality of edges. For example, distal tip 104 comprises 1, 2, 3, 4, 5, 6, 7, 8, 9, 10, or more than 10 edges, in various embodiments. In some embodiments, 1, 2, 3, 4, 5, 6, 7, 8, 9, 10, or more than 10 edges of distal tip 104 are sharp edges. In some embodiments, distal tip 104 comprises an edge (e.g., distal tip edge 144, distal tip edge 144a, or distal tip edge 144b) that is adjacent (e.g., as viewed in a coronal cross-section) to a side wall (e.g., surface 336, 336a, 336b, 338, 338a, and/or 338b) or edge (e.g., edge 332 or edge 334) of cutting head 100. In some embodiments, a point of distal tip 104 is formed where an edge of distal tip 104 (e.g., distal tip edge 144, distal tip edge 144a, or distal tip edge 144b) meets a side wall (e.g., surface 336, 336a, 336b, 338, 338a, and/or 338b) or edge (e.g., edge 332 or edge 334) of cutting head 100, for example, in a coronal plane 190 of distal tip 104 (e.g., as shown in FIG. 4F and FIG. 4H). In some embodiments, a distal tip edge 144 of distal tip 104 meets a (e.g., surface 336, 336a, 336b, 338, 338a, and/or 338b) or edge (e.g., edge 332 or edge 334) of cutting head 100 to form an angle omega (e.g., □□ of 1 degree to 90 degrees. In some embodiments, a distal tip edge 144 of distal tip 104 meets a side wall (e.g., surface 336, 336a, 336b, 338, 338a, and/or 338b) or edge (e.g., edge 332 or edge 334) of cutting head 100 to form an angle □ of 1 degree to 30 degrees, 1 degree to 45 degrees, 1 degree to 60 degrees, 1 degree to 90 degrees, 30 degrees to 45 degrees, 30 degrees to 60 degrees, 30 degrees to 90 degrees, 45 degrees to 60 degrees, 45 degrees to 90 degrees, 60 degrees to 90 degrees, from 90 degrees to 180 degrees, between 90 degrees and 135 degrees, or from 135 degrees to less than 180. In some embodiments, a distal tip edge 144 of distal tip 104 meets a side wall or edge of cutting head 100 (e.g., edge 332 or edge 334) to form an angle □ of 1 degree, 30 degrees, 45 degrees, 60 degrees, or 90 degrees. In some embodiments, a distal tip edge 144 of distal tip 104 meets a side wall or edge of cutting head 100 (e.g., edge 332 or edge 334) to form an angle □ of at least 1 degree, 30 degrees, 45 degrees, or 60 degrees. In some embodiments, a distal tip edge 144 of distal tip 104 meets a side wall or edge of cutting head 100 (e.g., edge 332 or edge 334) to form an angle □ of at most 30 degrees, 45 degrees, 60 degrees, or 90 degrees.

In some embodiments, a point of distal tip 104 is formed where a first edge of distal tip 104 (e.g., distal tip edge 144a) meets a second edge of distal tip 104 (e.g., distal tip edge 144b), for example, substantially within a coronal plane of distal tip 104 (e.g., as shown in FIG. 4G, FIG. 4I, and FIG. 4J). In some embodiments, a first distal tip edge 144a of distal tip 104 meets a second distal tip edge 144b of distal tip 104 (e.g., in a coronal plane of distal tip 104) to form an angle □ of 1 degree to 180 degrees. In some embodiments, a first distal tip edge 144a of distal tip 104 meets a second distal tip edge 144b of distal tip 104 (e.g., in a coronal plane of distal tip 104) to form an angle □ of 1 degree to 30 degrees, 1 degree to 45 degrees, 1 degree to 60 degrees, 1 degree to 90 degrees, 1 degree to 120 degrees, 1 degree to 135 degrees, 1 degree to 150 degrees, 1 degree to 180 degrees, 30 degrees to 45 degrees, 30 degrees to 60 degrees, 30 degrees to 90 degrees, 30 degrees to 120 degrees, 30 degrees to 135 degrees, 30 degrees to 150 degrees, 30 degrees to 180 degrees, 45 degrees to 60 degrees, 45 degrees to 90 degrees, 45 degrees to 120 degrees, 45 degrees to 135 degrees, 45 degrees to 150 degrees, 45 degrees to 180 degrees, 60 degrees to 90 degrees, 60 degrees to 120 degrees, 60 degrees to 135 degrees, 60 degrees to 150 degrees, 60 degrees to 180 degrees, 90 degrees to 120 degrees, 90 degrees to 135 degrees, 90 degrees to 150 degrees, 90 degrees to 180 degrees, 120 degrees to 135 degrees, 120 degrees to 150 degrees, 120 degrees to 180 degrees, 135 degrees to 150 degrees, 135 degrees to 180 degrees, or 150 degrees to 180 degrees. In some embodiments, a first distal tip edge 144a of distal tip 104 meets a second distal tip edge 144b of distal tip 104 (e.g., in a coronal plane of distal tip 104) to form an angle □ of 1 degree, 30 degrees, 45 degrees, 60 degrees, 90 degrees, 120 degrees, 135 degrees, 150 degrees, or 180 degrees. In some embodiments, a first distal tip edge 144a of distal tip 104 meets a second distal tip edge 144b of distal tip 104 (e.g., in a coronal plane of distal tip 104) to form an angle □ of at least 1 degree, 30 degrees, 45 degrees, 60 degrees, 90 degrees, 120 degrees, 135 degrees, or 150 degrees. In some embodiments, a first distal tip edge 144a of distal tip 104 meets a second distal tip edge 144b of distal tip 104 (e.g., in a coronal plane of distal tip 104) to form an angle □ of at most 30 degrees, 45 degrees, 60 degrees, 90 degrees, 120 degrees, 135 degrees, 150 degrees, or 180 degrees.

In some embodiments, a distal tip edge 144 of a pointed distal tip 104 is angled from a first lateral edge of the wire comprising the distal tip to a second lateral edge of the wire comprising the distal tip (e.g., as shown in FIG. 4F and FIG. 4H). In some embodiments, a distal tip edge 144 of a pointed distal tip 104 is angled with respect to a first side edge of the wire and a second side edge of the wire and extends past either the first side edge of the wire (e.g., as shown in FIG. 4I) or past the second edge of the wire (e.g., as shown in FIG. 4J). In some embodiments, such an angled distal tip edge 144 of pointed distal tip 104 extending beyond a first or second side edge of the wire of distal tip 104 (e.g., as shown in FIG. 4I and FIG. 4J, respectively) comprises a pointed tip useful for making cuts or incisions (e.g., into a capsule of an eye during an eye procedure). In many cases, one or more of distal tip edge 144, distal tip edge 144a, and/or distal tip edge 144b are sharp edges. For example, distal tip edge 144 is a sharp edge, in many cases. In many cases, distal tip edge 144a is a sharp edge. In many cases, distal tip edge 144b is a sharp edge. In some embodiments, distal tip edge 144a is a sharp edge and distal tip edge 144b is a sharp edge. In some embodiments, distal tip 104 has a curved shape (e.g., viewed in a coronal section of cutting head 100, for example, as shown in FIG. 4K). In some embodiments, a distal tip comprising a rounded tip allows for less complex manufacture.

In some embodiments, a distal tip 104 or a portion thereof is angled relative to an adjacent portion of cutting head 100 (e.g., first portion 108 and/or second portion 109, as shown in FIG. 2E, FIG. 2F, FIG. 2G, and FIGS. 4D, 4E, 4I, and 4J). In some embodiments, distal tip 104 or a portion thereof is angled relative to an adjacent portion of cutting head 100 at bend 232. In some embodiments, distal tip 104 is angled toward a medial surface 120 (e.g., away from lateral surface 122) of cutting head 100. In some embodiments, distal tip 104 is angled toward lateral surface 122 (e.g., away from medial surface 120) of cutting head 100.

In some embodiments, distal tip 104 or a portion thereof extends past an edge of the cutting head (e.g., edge 332 or edge 334), for example, as shown in FIG. 4I and FIG. 4J. In some embodiments, a deflection distance 226 of a distal tip past an edge of the cutting head is 1% to 500% of a width 216 of the cutting head. In some embodiments, a deflection distance 226 of a distal tip past an edge (e.g., edge 332 or edge 334) of the cutting head is 1% to 10%, 1% to 50%, 1% to 100%, 1% to 200%, 1% to 300%, 1% to 400%, 1% to 500%, 10% to 50%, 10% to 100%, 10% to 200%, 10% to 300%, 10% to 400%, 10% to 500%, 50% to 100%, 50% to 200%, 50% to 300%, 50% to 400%, 50% to 500%, 100% to 200%, 100% to 300%, 100% to 400%, 100% to 500%, 200% to 300%, 200% to 400%, 200% to 500%, 300% to 400%, 300% to 500%, or 400% to 500% of a width 216 of the cutting head (e.g., as measured within the coronal plane of the distal tip of the cutting head). In some embodiments, a deflection distance 226 of a distal tip past an edge of the cutting head is 1%, 10%, 50%, 100%, 200%, 300%, 400%, or 500% of a width 216 of the cutting head. In some embodiments, a deflection distance 226 of a distal tip past an edge of the cutting head is at least 1%, 10%, 50%, 100%, 200%, 300%, or 400% of a width 216 of the cutting head. In some embodiments, a deflection distance 226 of a distal tip past an edge of the cutting head is at most 10%, 50%, 100%, 200%, 300%, 400%, or 500% of a width 216 of the cutting head.

In an exemplary embodiment of the invention, cutting head 100 comprises a super elastic rod that is entered into the eye and formed into a predetermined shape within the eye. In some embodiments, the oscillation is activated after the predetermined shape is formed. In some embodiments of the invention, the predetermined shape comprises a circle with a predetermined radius, such as described in U.S. Pat. No. 6,551,326. Alternatively or additionally, any other method of adjusting the shape of the cutting head within the eye is performed before applying the oscillation.

### Cutting Head Dimensions

In some embodiments, all or a portion of first portion 108 comprises a curve. In some embodiments, a curved portion of cutting head 100 comprises a sharp edge and/or a pointed tip. In some embodiments, a curved portion of cutting head 100 comprises a plurality of sharp edges (e.g., 2 sharp edges or more than 2 sharp edges). A curved portion of cutting head 100 (e.g., all or a portion of first portion 108) can have a radius of curvature 112. A radius of curvature of cutting head 100 (or a portion thereof, such as all or a portion of first portion 108) is at least 1 mm, at least 2 mm, at least 3 mm, at least 4 mm, at least 5 mm, at least 6 mm, at least 7 mm, at least 8 mm at least 9 mm, at least 10 mm, more than 10 mm, from 1 mm to 10 mm, from 2 mm to 9 mm, from 2 mm to 8 mm, from 2 mm to 7 mm, from 2 mm to 6 mm, from 2 mm to 5 mm, from 2 mm to 4 mm, or from 2 mm to 3 mm, in some embodiments. In some embodiments, a curved portion of cutting head 100 has a radius of curvature of 2.75 mm A curved portion of cutting head 100 has a diameter of at least 1 mm, at least 2 mm, at least 3 mm, at least 4 mm, at least 5 mm, at least 6 mm, at least 7 mm, at least 8 mm at least 9 mm, at least 10 mm, more than 10 mm, from 1 mm to 10 mm, from 2 mm to 9 mm, from 3 mm to 8 mm, from 4 mm to 7 mm, or from 5 mm to 6 mm, in some embodiments. In some embodiments, a curved portion of cutting head 100 has a diameter of 5.5 mm. In some embodiments, a curve of cutting head 100 comprises a circular arc of less than 360 and more than 0 degrees. In many cases, a curve of cutting head 100 comprises a circular arc of 30 to 270 degrees, 45 degrees to 225 degrees, 60 degrees to 215 degrees, or 90 degrees to 180 degrees. In some embodiments, a curve of cutting head 100 comprises a circular arc of exactly 180 degrees. In some embodiments, a cutting head comprising a portion curved into a circular arc of 180 degrees and having a diameter of 5.5 can be well-suited for use in eye procedures, such as cataract surgery (e.g., on an adult patient). In some embodiments, a curve of cutting head 100 comprises a circular arc. In some embodiments, a curve of cutting head 100 comprises an elliptical arc. In some embodiments, a curve of cutting head 100 comprises an ellipsoid arc. In some embodiments, a curve of cutting head 100 comprises an oval arc. In some embodiments, a curve of cutting head 100 comprises an ovoid arc. In some embodiments, a curve of cutting head 100 comprises a variable radius arc that is non-circular. In some embodiments, a curve of cutting head 100 comprises a non-straight path along its length from the proximal end to the tip of the cutting head.

In some embodiments, a width 216 of a cutting head 100 (or a portion of cutting head 100) is at least 1 mm, at least 2 mm, at least 3 mm, at least 4 mm, at least 5 mm, at least 6 mm, at least 7 mm, at least 8 mm at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, more than 20 mm, from 1 mm to 20 mm, from 3 mm to 17 mm, from 5 mm to 15 mm, from 7 mm to 13 mm, or from 9 mm to 11 mm. In some embodiments, the width of a cutting head tapers after a shoulder point 220 (e.g., as shown in FIGS. 2C and 2E). In some embodiments, a width 216 of a cutting head 100 is constant over a portion of a cutting head 100.

A medial surface 120 of cutting head 100 (or a portion of cutting head 100) has a width 306 of at least 1 mm, at least 2 mm, at least 3 mm, at least 4 mm, at least 5 mm, at least 6 mm, at least 7 mm, at least 8 mm at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, more than 20 mm, from 1 mm to 20 mm, from 3 mm to 17 mm, from 5 mm to 15 mm, from 7 mm to 13 mm, or from 9 mm to 11 mm, in some embodiments. In some embodiments, a lateral surface 122 of cutting head 100 (or a portion of cutting head 100) has a width 304 of at least 1 mm, at least 2 mm, at least 3 mm, at least 4 mm, at least 5 mm, at least 6 mm, at least 7 mm, at least 8 mm at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, more than 20 mm, from 1 mm to 20 mm, from 3 mm to 17 mm, from 5 mm to 15 mm, from 7 mm to 13 mm, or from 9 mm to 11 mm.

In some embodiments, a width of lateral surface 122 is larger than a width of medial surface 120 over at least a portion of a cutting head 100 (e.g., as measured within a cross-sectional plane perpendicular to the cutting head wire), for example, as shown in FIGS. 3B-3D. In some embodiments, a width of medial surface 120 is equal to a width of lateral surface 122 over at least a portion of a cutting head 100 (e.g., as measured within a cross-sectional plane perpendicular to the cutting head wire), for example, as shown in FIG. 3E. In some embodiments, a width of medial surface 120 is larger than a width of lateral surface 122 over at least a portion of a cutting head 100 (e.g., as measured within a cross-sectional plane perpendicular to the cutting head wire), for example, as shown in FIGS. 3G-3I. In some embodiments, a medial surface 120 is an edge over at least a portion of a cutting head 100 (e.g., as measured within a cross-sectional plane perpendicular to the cutting head wire), for example, as shown in FIG. 3A. That is, in some embodiments, medial surface 120 (or at least a portion thereof) of a cutting head 100 has a width of 0.0 mm. In some embodiments, a medial surface 120 is an edge and a lateral surface 122 of a wire of cutting head 100 comprises a surface over at least a portion of a cutting head 100 (e.g., as measured within a cross-sectional plane perpendicular to the cutting head wire). That is, in some embodiments, medial surface 120 has a width of 0.0 mm and lateral surface 122 has a width of greater than 0.0 mm over at least a portion of a cutting head 100 (e.g., as measured within a cross-sectional plane perpendicular to the cutting head wire). In some embodiments, a lateral surface 122 (or at least a portion thereof) of a cutting head 100 is an edge, for example, as shown in FIG. 3F. That is, in some embodiments, lateral surface 122 (or at least a portion thereof) of a cutting head 100 has a width of 0.0 mm. In some embodiments, a lateral surface 122 is an edge and a medial surface 120 comprises a surface over at least a portion of a cutting head 100 (e.g., as measured within a cross-sectional plane perpendicular to the cutting head wire). That is, in some embodiments, lateral surface 122 has a width of 0.0 mm and medial surface 120 has a width greater than 0.0 mm over at least a portion of a cutting head 100 (e.g., as measured within a cross-sectional plane perpendicular the cutting head wire).

In some embodiments, a width of lateral surface 122 is larger than a width of medial surface 120 over at least a portion of a curved portion of cutting head 100 (e.g., as measured within a cross-sectional plane perpendicular to the curvature of the portion of the cutting head). In some embodiments, a width of medial surface 120 is equal to a width of lateral surface 122 over at least some of a curved portion of cutting head 100 (e.g., as measured within a cross-sectional plane perpendicular to the curvature of the curved portion of the cutting head). In some embodiments, a width of medial surface 120 is larger than a width of lateral surface 122 of at least some of a curved portion of cutting head 100 (e.g., as measured within a cross-sectional plane perpendicular to the curvature of the curved portion of the cutting head). In some embodiments, a medial surface 120 of a curved portion of cutting head 100 is an edge. In some embodiments, a medial surface 120 is an edge and a lateral surface 122 of a wire of cutting head 100 comprises a surface over at least some of a curved portion of a cutting head 100 (e.g., as measured within a cross-sectional plane perpendicular to the curvature of the curved portion of the cutting head). That is, in some embodiments, medial surface 120 has a width of 0.0 mm and lateral surface 122 has a width of greater than 0.0 mm over at least some of a curved portion of a cutting head 100 (e.g., as measured within a cross-sectional plane perpendicular to the curvature of the curved portion of the cutting head). In some embodiments, a lateral surface 122 of a curved portion of cutting head 100 is an edge. In some embodiments, a lateral surface 122 is an edge and a medial surface 120 of a wire of cutting head 100 comprises a surface over at least some of a curved portion of a cutting head 100 (e.g., as measured within a cross-sectional plane perpendicular to the curvature of the curved portion of the cutting head). That is, in some embodiments, lateral surface 122 has a width of 0.0 mm and medial surface 120 has a width greater than 0.0 mm over at least some of a curved portion of a cutting head 100 (e.g., as measured within a cross-sectional plane perpendicular to the curvature of the curved portion of the cutting head).

A cutting head 100 (or a portion thereof) has a thickness 222 of at least 0.1 mm, at least 0.5 mm, at least 1 mm, at least 1.5 mm, at least 2 mm, at least 3 mm, at least 4 mm, at least 5 mm, less than 0.1 mm, from 0.1 mm to 5 mm, from 0.1 mm to 4 mm, from 0.1 mm to 3 mm, from 0.1 mm to 2 mm, from 0.5 mm to 1.5 mm, or from 0.75 mm to 1.25 mm, from 0.1 mm to 1 mm, from 0.25 mm to 0.75 mm, from 0.4 mm to 0.6 mm, or less than 0.1 mm, in some embodiments. In some embodiments, the thickness 222 of cutting head 100 is constant from a proximal end of a cutting head 100 to a distal tip 104. In some embodiments, the thickness 222 of cutting head 100 is not constant from a proximal end of cutting head 100 to a distal tip 104.

In some embodiments of the invention, a semi-circular portion of cutting head 100 has a diameter of at least 1 mm, at least 2 mm, at least 3 mm, at least 4 mm, at least 5 mm, at least 6 mm, at least 7 mm, at least 8 mm at least 9 mm, at least 10 mm, more than 10 mm, from 1 mm to 10 mm, from 2 mm to 9 mm, from 3 mm to 8 mm, from 4 mm to 7 mm, or from 5 mm to 6 mm. In some embodiments, a curved portion of cutting head 100 has a diameter of 5.5 mm. In some embodiments, a semi-circular portion of cutting head 100 has a diameter of between about 6.5-7.5 millimeters (mm). Cutting head 100 is optionally larger than the size of a required incision, by between about 10-20%, or even 40-50%, as the area of the cut is defined by the crossing of the curved lines cut out by both of sharp edges 332 and 334. Alternatively, cutting head 100 has a smaller size, optionally having a diameter smaller than 5 millimeters or even smaller than 2 millimeters. In an exemplary embodiment of the invention, cutting head 100 has a diameter of between about 0.5-1.5 millimeters. Such small cutting heads are optionally used in procedures in which the lens of the eye is removed by liquidification or partial liquidification and therefore only a small incision is required.

A cutting device comprises a handle connector 127 for attaching cutting head 100 to cutting device body 102, in some embodiments. In many cases, a primary function of a handle connector 127 is to prevent the cutting head 100 from separating from and/or moving relative to cutting device body 102 or a portion thereof. In many cases, a handle connector 127 prevents cutting head 100 from rotating about a longitudinal axis 140 (e.g., in direction 142) relative to cutting device body 102 or a portion thereof (e.g., housing 123, spindle 133, oscillator 131, or shaft 132). In many cases, a handle connector 127 prevents cutting head 100 from moving in an axial direction (e.g., either proximally or distally along a longitudinal axis) relative to cutting device body 102 or a portion thereof (e.g., housing 123, spindle 133, oscillator 131, or shaft 132). By preventing cutting head 100 from separating from and/or moving relative to cutting device body 102 or a portion thereof, it is possible to make cuts and incisions with the cutting device that are required for many medical procedures, such as eye procedures like cataract surgery.

Various mechanisms and strategies are used to attach cutting head 100 to cutting device body 102 in embodiments disclosed herein, including chemical fixation (e.g., the use of adhesives like a glue or epoxy), physical joining (e.g., soldering or melting two components together), or mechanical fixation (e.g., mechanical fixtures or fasteners such as clips, spring clips, retainer clips, screws, nut and bolt systems, rivets, pins, friction joints, or clamps, such as a band clamp). In many cases, an interface between a cutting head 100 or portion thereof (e.g., cutting head handle 114) and handle connector 127 comprises a metal-to-metal interface, a metal-to-plastic interface, or a plastic-to plastic interface. For example, the interface between a cutting head 100 or a portion thereof (e.g., cutting head handle 114) and the handle connector 127 is a metal-to-metal interface (e.g., if the cutting head handle is metal and the handle connector 127 or a portion thereof is metal), in some embodiments. In some embodiments, the interface between a cutting head 100 or a portion thereof and the handle connector 127 is a metal-to-plastic interface (e.g., if the cutting head handle is metal and the handle connector 127 is plastic). In some embodiments, devices and systems disclosed herein comprising a material with low mechanical compliance (e.g., metals and/or plastics with a high stiffness, for example having a Young's modulus greater than 3.0 GPa at room temperature) can aid in improving cutting head-to-handle connector joint strength, improving efficiency of energy transfer from cutting device body 102 or a portion thereof (e.g., oscillator 131) to the cutting head 100 or a portion thereof (e.g., a sharp edge of cutting head 100), and reducing undesired rotation or axial motion of the cutting head relative to the cutting device body 102 or a portion thereof.

In some embodiments, a handle connector 127 comprises a permanent (e.g., irreversible) joint with a cutting head 100 or portion thereof (e.g., handle 114).

In some embodiments, a handle connector 127 comprising a permanent or irreversible joint can be advantageous, for example, as permanently joining a cutting head to a cutting device body 102 can improve the strength and/or stability of the cutting head during operation and/or reduce the effect of vibrations on the strength of a reversible joint over many uses. Furthermore, conduction of vibrational energy from an oscillator to a sharp edge of a cutting head 100 is often improved in cases where the cutting head 100 is permanently joined to the cutting device body 102.

In some embodiments, cutting head 100 or a portion thereof (e.g., handle 114) is rigidly coupled to a portion of a cutting device body 102, such as handle connector 127, oscillator 131, or shaft 132. Handle connectors 127 useful in rigidly coupling a cutting head 100 (e.g., cutting handle 114) to cutting device body 102 include chemical connectors (e.g., glues, epoxies, silicone-based adhesives, cyanoacrylates, urethane adhesives, and polyurethane adhesives), tapes (e.g., duct tape, electrical tape, friction tape, gaffer tape, surgical tape, PTFE tape, or metal tape), physical joining (e.g., soldering, welding, or melting two components together), or mechanical fixation (e.g., fasteners such as clips, spring clips, retainer clips, screws, nut and bolt systems, rivets, pins, friction joints, or clamps, such as a band clamp).

In many cases, handle connector 127 can improve the efficiency of energy transfer (e.g., vibrational energy) from an oscillator to cutting head 100 or a portion thereof (e.g., a sharp edge of cutting head 100) or from a surgeon's hand to cutting head 100 or a portion thereof via cutting device body 102. In some embodiments, a rigid connection between cutting head 100 or a portion thereof and a portion of cutting device body 102 can improve the efficiency of energy transfer during operation of the cutting device (e.g., transfer of vibrational energy from an actuation mechanism, such as an oscillator). For example, in some embodiments, loss of energy (e.g., oscillatory energy, such as vibrational energy, rotational energy, linear actuation energy, from an oscillator) due to friction or compliance of a joint of the cutting device can be minimized by increasing the rigidity with which two or more portions of the cutting device are coupled to one another.

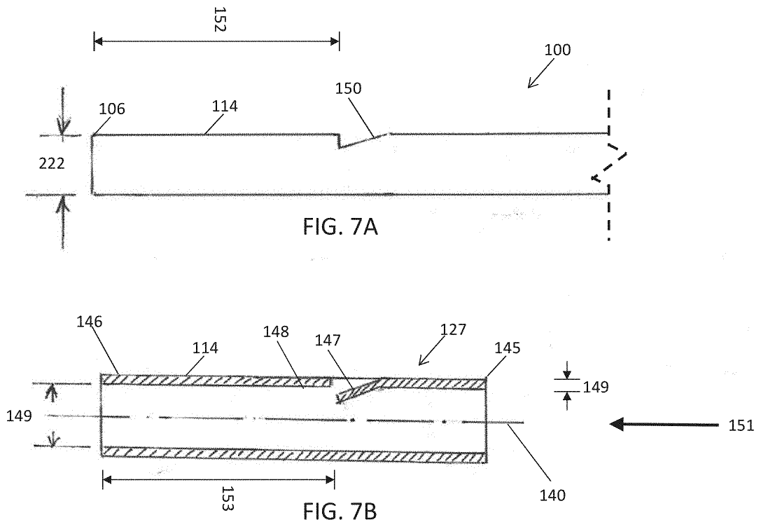

An exemplary handle connector 127 is shown in FIG. 7B. In many embodiments, handle connector 127 comprises a shaft having an inner surface 148 and a distal end 145. In many embodiments, inner surface 148 of handle connector 127 has a shape substantially the same as those of cutting head handle 114. Inner dimension 149 of handle connector 127 is substantially the same as a dimension of handle 114 (e.g., width 216, thickness 222, width 304, or width 306) or slightly larger than (e.g., from 0.5% to 5%, from 5% to 10%, from 10% to 20%, or from 20% to 40% larger than) the dimension of handle 114, in many cases, for example, to ensure a secure fit of handle 114 in handle connector 127. In some embodiments, handle connector 127 comprises handle fixture flange 147. Handle fixture flange 147 comprises a portion of the wall 148 of handle connector 127 that is angled into the shaft of handle connector 127, in many embodiments. In many cases, handle fixture flange 147 is angled inward toward longitudinal axis 140 and toward a proximal end 146 of handle connector 127 so that, when a cutting head 100 with a handle fixture notch 150 (e.g., as shown in FIG. 7A) is inserted into handle connector 127 (e.g., in direction 151), handle fixture flange 147 of handle connector 127 slots into handle fixture notch 150 of cutting head 100, for example, to secure handle 114 of cutting head 100 in handle connector 127.

In many embodiments, the distance 152 between a proximal end of handle fixture notch 150 and a proximal end of cutting head handle 114 is sized relative to the distance 153 between a proximal end of handle fixture flange 147 and a proximal end of handle connector 127 (which comprises a solid wall or directly abuts a solid surface of oscillator 131, such as shaft 132, in some embodiments) to prevent axial motion of cutting head handle 114 within handle connector 127. For example, distance 152 is the same as distance 153, in some embodiments. In some embodiments, distance 152 is not exactly the same as distance 153 but is substantially the same and still prevents substantial axial motion of cutting head handle 114 within handle connector 127.

In some embodiments, rotational motion of cutting head handle 114 within handle connector 127 is prevented by the relative shapes of the cutting head handle 114 and the inner wall 148 of handle connector 127. For example, cutting head handle 114 is rectangular or square in cross-section (e.g., transverse cross-section) and inner wall 148 of handle connector 127 is rectangular or square in cross-section (e.g., transverse cross-section), in many cases. When cutting head handle 114 has similar cross-sectional dimensions and shape (e.g., rectangular, square, or another non-circular shape) as the inner wall 148 of handle connector 127, rotation of cutting head handle 114 is prevented by the shape of the cutting head handle 114 within the handle connector 127. The rotational stability of cutting head handle 114 within handle connector 127 can be further improved by employing a flange/notch system such as the embodiments illustrated in FIGS. 7A and 7B, even when the relative shapes and dimensions of the cutting head handle 114 (or a portion thereof) and inner wall 148 of handle connector 127 substantially inhibit rotation of cutting head handle 114 within handle connector 127. Furthermore, a flange/notch system, such as is shown in FIGS. 7A and 7B, prevents axial motion of cutting head handle 114 within handle connector 127, as described above.

In some embodiments, one or both of cutting head handle 114 and inner wall 148 of handle connector 127 have substantially circular transverse cross-sections. In such cases, inclusion of handle fixture notch 150 in cutting head handle 150 and handle fixture flange 147 in handle connector 127 is advantageous for preventing rotation of cutting head handle 114 (e.g., about longitudinal axis 140) within handle connector 127. For example, handle fixture flange 147 can have a width (e.g., oriented into and/or out of the plane of the page of FIG. 7B) that prevents rotation of cutting head handle 114 (e.g., around longitudinal axis 140) relative to handle connector 127 when interfaced with handle fixture notch 150 of cutting head handle 114.

In some embodiments, handle connector 127 comprises an adjustable or reversible joint with a cutting head 100 or portion thereof (e.g., handle 100). A handle connector 127 comprising an adjustable or reversible joint can be advantageous, for example, in cases where a cutting device is used for multiple eye procedures (e.g., which may require differently sized cutting heads or cleaning of cutting heads) or where a cutting device is used for multiple functions in a single procedure. In some embodiments, a handle connector 127 comprising an adjustable or reversible joint comprises a release mechanism, such as a release lever or a release button configured to release the cutting head 100 or a portion thereof (e.g., cutting head handle 114) from the handle connector 127.

### Cutting Head Actuation

FIG. 5 is an enlarged view of oscillator 108 and cutting head 100 placed against an eye 510, in accordance with an exemplary embodiment of the invention. Oscillator 108 optionally comprises a motor 130 which pushes cutting head 100 distally, and a spring 112, which retracts cutting head 100 proximally, so as to generate the oscillating movement. Optionally, motor 130 includes a piezoelectric crystal. Alternatively or additionally, any oscillation method and/or apparatus known in the art, may be used for inducing the oscillations of cutting head 100, for example apparatus used for power-driven toothbrushes. Some of such mechanisms are described, for example, in U.S. Pat. No. 6,845,537 and/or U.S. Pat. No. 6,371,294.

Alternatively or additionally, motor 130 can comprise a small eccentric motor (e.g., a motor with an off-axis weight) with an attached mass which moves the center of mass of the motor away from the central axis of the motor. Further alternatively or additionally, an oscillator as used in portable cellular telephones can be used instead of or in addition to motor 130.

In some embodiments of the invention, cutting device 102 is configured to cause cutting head 100 or a portion thereof to oscillate (e.g., vibrate) at a rate of at least 10 Hz, at least 20 Hz, at least 50 Hz, at least 100 Hz, at least 200 Hz, at least 300 Hz, at least 400 Hz, at least 500 Hz, at least 1000 Hz, at least 1500 Hz, at least 2000 Hz, at least 2500 Hz, at least 3000 Hz, at least 3500 Hz, at least 4000 Hz, at least 4500 Hz, or at least 5000 Hz when in operation. In some embodiments, cutting device 102 can be configured to cause cutting head 100 or a portion thereof to oscillate (e.g., vibrate) at a rate of 10 Hz to 100 Hz, 100 Hz to 500 Hz, 300 Hz to 500 Hz, 500 Hz to 1000 Hz, 1000 Hz to 2000 Hz, 2000 Hz to 3000 Hz, 3000 Hz to 4000 Hz, 4000 Hz to 5000 Hz, or more than 5000 Hz. Optionally, cutting head 100 can oscillate at a rate of at most 5000 Hz, at most 4000 Hz, at most 3000 Hz, at most 2000 Hz, at most 1000 Hz, at most 500 Hz, at most 300 Hz, less than 100 Hz, less than 50 Hz or even less than 30 Hz. Alternatively, cutting head 100 can oscillate at higher or lower rates. In some embodiments of the invention, the rate of oscillation of cutting head 100 is adjustable by a physician or technician, according to personal preferences and/or the texture of tissue being cut. The oscillation rate is optionally selected to achieve the cut of the tissue while requiring from a physician minimal pressure of the cutting head against the tissue. In some embodiments, increasing a rate of oscillation (e.g., vibration) of a cutting head 100 or portion thereof can improve function of the device or system during a procedure, for example, by minimizing damage to tissue when the cutting head is applied to the tissue.

The amplitude of the oscillations of cutting head 100 can be, for example, at least 0.02 mm or at least 0.1 mm. In some embodiments of the invention, the amplitude of the oscillations may be smaller than 1 millimeter or even smaller than 0.5 millimeters. In an exemplary embodiment of the invention, the amplitude of the oscillations is smaller than 0.2 millimeters.

In some embodiments, those portions of cutting head 100 which are relatively parallel to the direction of oscillation, operate as a saw when the oscillating is performed. The parallel portions generally cut into the tissue in a first stage of the cutting. The movement of the parallel portions of cutting head 100 downward into the cut tissue, possibly causes the portions of cutting head 102 that are perpendicular to the oscillation to cut into the tissue as they decline into the tissue with the parallel portions.

Alternatively or additionally to oscillating along the axis of cutting device 100, cutting head 100 oscillates in other directions, for example in a direction indicated by an arrow 111, in the plane of the axis of the cutting device perpendicular to the axis. In some embodiments of the invention, the oscillation is in a diagonal direction, for example 45° to the axis of the cutting device. In some embodiments of the invention, the direction of the oscillation changes during a cutting procedure, so that different portions of cutting head 100 have a saw effect on the tissue. In some embodiments of the invention, cutting head 100 oscillates in a direction normal to the cut surface, or in a diagonal direction having a component normal to the cut surface.

In some embodiments of the invention, cutting head 100 and handle 114 are permanently mounted on housing 123. Alternatively, housing 123 includes a receptacle adapted to receive a manual prior art cutting device and to provide oscillation to the cutting head. Further alternatively, handle 114 detachably connects to cutting head 100, allowing, for example, mounting of different size cutting heads 100 on handle 114 and/or replacement for sterilization of the cutting heads.

Alternatively to requiring that a physician turn over the cutting head by turning over the cutting device, an automatic mechanism is provided within the cutting device for flipping the cutting head, as is now described in relation to FIG. 5.

FIG. 1J is a schematic sectional view of a cutting device, in accordance with an exemplary embodiment of the invention. In some embodiments, a cutting device comprises a cutting head 502 with a handle 504 extending within a housing 520. A blade cover 530 can be used to cover a blade of cutting head 502 and hence prevent inadvertent cutting by the blade. As shown in FIG. 1J, blade cover 530 can be half employed, covering only a proximal half of cutting head 502. A cover manipulation handle 534 can be moved axially in parallel to handle 504, in order to remove blade cover 530 from covering cutting head 502 and/or in order to cover cutting head 502. Moving handle 534 proximally, as indicated by an arrow 532, can expose the blades of cutting head 502 for cutting, while moving handle 534 distally, in the direction opposite that indicated by arrow 532, conceals the blade of cutting head 502. Blade cover 530 optionally comprises a semi-rigid plastic, which on the one hand moves without folding over itself, and on the other hand can follow the contours of cutting head 502. Alternatively, blade cover 530 can be formed of any other material that allows movement back and forth to conceal and expose the blades of cutting head 502.

In some embodiments of the invention, cutting head 502 is inserted into the patient's eye with blade cover 530 completely covering cutting head 502. When cutting head 502 is in place, blade cover 530 can be retracted and the cutting can be performed. When the cutting is completed, blade cover 530 can be moved back to cover cutting head 502 and the cutting head can be removed from the patient's eye. In some embodiments of the invention, cutting head 502 is also covered before flipping cutting head 502 within the patient's eye. Alternatively, blade cover 530 can be moved within the patient's eye only proximally (or only distally). In accordance with this alternative, the production of blade cover 530 can be simpler in some embodiments.

An oscillation motor 526 optionally oscillates cutting head 502 under control of a button (or other control) 518.

In some embodiments of the invention, a pivot 524 controlled by a rotation motor 514 is used by the physician to rotate handle 504 and hence cutting head 502. In some embodiments of the invention, the rotation of pivot 524 is used to flip cutting head 502, instead of turning housing 520. Optionally, rotation motor 514 is actuated by a button 516. In some embodiments of the invention, the actuating of button 516 causes pivot 524 to rotate 180°. Alternatively, actuating of button 516 can cause pivot 524 to rotate in small steps (e.g., 15°, 30°, 45°, 60°), allowing the physician to control the angle of the cutting head. Further alternatively, pivot 524 can rotate in a continuous manner when button 516 is actuated.

Alternatively to a rotation motor 514, pivot 524 is controlled mechanically, for example by a lever directly attached to pivot 524, which is rotated by the physician. In some embodiments of the invention, rather than being battery operated, cutting head 100 is operated by a power cable or other energy source.

While the above description relates to a capsulotomy cutting device, the device of the present invention may be used to cut tissue in other body organs, such as the brain, head or neck, especially where it is desired to cut a circular, semi-circular or other planar cut beneath tissue, using an access hole smaller than the desired cut.

It will be appreciated that the above described methods of using the cutting device may be varied in many ways, including performing three or more cuts in achieving an incision. In some embodiments of the invention, however, a complete incision is achieved with no more than ten or even five placements of cutting head 100 against different locations on the lens capsule. It should also be appreciated that the above described description of methods and apparatus are to be interpreted as including apparatus for carrying out the described methods and methods of using the described apparatus.

### Protective Cover

In some embodiments of the invention, a protective cover (not shown) is slid over cutting head 100 when not in use. Alternatively or additionally, the protective cover covers cutting head 100, while the cutting head is maneuvered into the anterior chamber of a subject's eye. In some embodiments of the invention, the cover is connected to a control on housing 123, which allows a physician to remove the cover, while cutting head 100 is within the anterior chamber. Alternatively or additionally, the physician can move the cover back onto cutting head 100, while cutting head 100 is within the anterior chamber. Optionally, the control comprises a thin string running along or within housing 123.

### Cauterizing Cutting Head

In some embodiments the device comprises a cauterizer. The device may, in such embodiments comprise a cauterizing cutting head. In any of the embodiments described or shown in any figure herein, for example, the cutting head 100, or a portion thereof, may be configured using a thermal cauterizer or other cauterizing means. The source of cautery may be an electrical or other energy source, which cauterizes tissue. In such embodiments, the device or devices may be referred to herein as having a cutting head 100 or a cauterizing cutting head. The cutting head 100 may include, in any embodiment herein, a connection to an energy source that transfers the energy from the energy source to the cutting head 100 or a portion thereof sufficient to and adapted to cauterize the tissue upon contact therewith or within a short period of contact therewith, such as within about 0.25 seconds, within about 0.5 seconds, within about 1 second, within about 2 seconds, within about 3 seconds, within about 4 seconds, within about 5 seconds, within at most about 5 seconds, within at most about 10 seconds, within at most about 15 seconds, within at most about 30 seconds. In many embodiments, a first portion 108 comprises a plurality of edges 332, 334. In some embodiments one or more edges are sharp or blade edges. In some embodiments, a cutting head 100 of a cutting device comprises a distal tip 104, a first portion 108, a proximal end 106, and a handle 114. In some embodiments the first portion 108 includes an edge 332 that is sharp or is a blade. In some embodiments the first portion 108 of cutting head 100 comprises a first edge (e.g., edge 332) and a second edge (e.g., edge 334), both of which are sharp or blade edges. Conduction or an electrical coupling may be used to transfer the energy from the energy source to the cutting head. In some embodiments, the curved first portion 108, one or more edges 332, 334, the tip 104, or any combination of these may be coupled to one or more energy source for cauterization of the tissue. In such cauterizing embodiment or embodiments, the cutting head comprises a connection to a radio frequency (“RF”) energy source (e.g. such as an electrical current generator), or a connection to an electrical source (e.g. a battery or other electrical source such as wall power source), or any other energy source. In a RF embodiment, the RF energy source is an electrical current generator and the cutting head or a portion thereof is an electrode in a monopolar or bipolar configuration. In a bipolar embodiment, the active electrode is the cutting head or a portion thereof, such as the curved first portion 108, either or both blades 332, 334, or the tip 104, and is used with a separate electrode (return electrode) on an opposite side of the tissue to be cauterized to complete the electrical circuit. In a monopolar RF embodiment cauterizing cutting head, the active electrode is the cutting head or a portion thereof, such as the curved first portion 108, either or both blades 332, 334, or the tip 104, the patient return electrode (e.g. dispersive pad) is placed on the patient body. In an electrocautery embodiment where direct or alternating current is passed from the energy source such as a battery or wall power source through a conductive wire to the curved first portion 108, either or both blades 332, 334, the tip 104, or a combination thereof. In an electrocautery embodiment, the device may comprise monopolar electrocauterizer, or a bipolar electrocauterizer. Any of the energy sources (whether RF generator or other energy source) may be configured, through circuitry or one or more connections to the cutting head 100 or any portion thereof, or both circuitry and a connection to the cutting head 100 or any portion thereof, to transfer energy (in the form of heat) from the energy source to tissue sufficient to cauterize such tissue. The temperature setpoint of the device may be up to or about 400 degrees Fahrenheit (“° F.”), up to or about 350° F., up to or about 450° F., up to or about 500° F., up to or about 700° F. up to or about 800° F., up to or about 900° F., up to or about 1000° F., up to or about 1100° F., up to or about 1200° F., up to or about 1300° F., up to or about 1400° F., up to or about 1500° F., up to or about 1600° F., up to or about 1700° F., up to or about 1800° F., up to or about 1900° F., up to or about 2000° F., up to or about 2300° F., up to or about 2500° F., or any range within or up to these setpoints. The temperature of the curved first portion 108, either or both blades 332, 334, the tip 104, or a combination thereof of the device may be up to or about 400° F., up to or about 450° F., up to or about 500° F., up to or about 700° F. up to or about 800° F., up to or about 900° F., up to or about 1000° F., up to or about 1100° F., up to or about 1200° F., up to or about 1300° F., up to or about 1400° F., up to or about 1500° F., up to or about 1600° F., up to or about 1700° F., up to or about 1800° F., up to or about 1900° F., up to or about 2000° F., up to or about 2300° F., up to or about 2500° F. or any range within or up to these setpoints. Further, the energy source, the temperature setpoint, or the temperature of the curved first portion 108, either or both blades 332, 334, the tip 104, or a combination thereof may be modulated or controlled through circuitry and one or more control such as a button, switch, control panel (computer generated manipulatable display or mechanical) or foot pedal that allows for on/off or controlled variable adjustment of the cauterization aspect of the device, at least. The control may provide a dial or other mechanical or electromechanical feature that allows for a range of energy delivery to the cutting head. The range may be a discrete range of temperature or energy setpoints from off to a minimum energy or minimum temperature, through a maximum energy or maximum temperature that cauterizes tissue. The range may be an analog range of the energy or temperature delivered to the cutting head or portion(s) thereof. The setpoints or range may be from off to high, or from 0 to 10, or from a minimum to a maximum temperature which may be a setpoint or a function of time at a setpoint, or otherwise calibrated and/or controlled. The device may include a temperature sensor on the curved first portion, the cutting head, or any portion thereof. The temperature sensor may provide feedback to the circuitry and include a readout of actual temperature to the user in a display or connection to a display. The circuitry may cut off the energy source or reduce the energy delivered to the cutting head (or any portion thereof) above a safe range, i.e. above a maximum temperature. The safe range or maximum energy or temperature may be pre-set, or be based on a control setting chosen by a user. The sensor and circuitry may automatically turn off or reduce the energy delivery, and may automatically resume upon temperature reduction at the sensor to a pre-set safe range or based on a chosen safe range based on the pre-set settings of the temperature or based on the user input of the controls. The safe range may be within about 1 degree Fahrenheit (“° F.”) of a setpoint temperature, within about 2° F. of a setpoint temperature, within about 3° F. of a setpoint temperature, within about 4° F. of a setpoint temperature, within about 5° F. of a setpoint temperature, within about 6° F. of a setpoint temperature, within about 7° F. of a setpoint temperature, within about 8° F. of a setpoint temperature, within about 9° F. of a setpoint temperature, within about 10° F. of a setpoint temperature, within about 15° F. of a setpoint temperature, within about 20° F. of a setpoint temperature, within about 25° F. of a setpoint temperature, within about 30° F. of a setpoint temperature, within about 40° F. of a setpoint temperature, within about 50° F. of a setpoint temperature, within about 60° F. of a setpoint temperature, within about 75° F. of a setpoint temperature, within about 100° F. of a setpoint temperature. The setpoint temperature may be the maximum setpoint in a range that is preset or chosen by a user. The control of the cauterization aspects of the device may be separate from the control of the vibration of the cutting head, and may be separately or simultaneously directed using the control of either or both. The device may include a mode switch configured to allow a user to choose between off, cauterization alone, vibration alone, or simultaneous cauterization and vibration, with the appropriate circuitry and programming to allow for each mode. Thus, in embodiments comprising a cauterizing cutting head, the device comprises circuitry that couples to the cutting head or any portion thereof thereby transferring a controlled source of cautery to the tissue. The same energy source may be used to move the cutting head 100 as is used to cauterize the tissue through the tip 104 or the first portion 108, the first blade 332, the second blade 334, or any combination thereof. The device may include shielding or electrical insulation on any portion of the cutting head 100. In some embodiments, the shielding or electrical insulation is on the handle 114 between the first portion and the proximal end. In some embodiments, shielding or electrical insulation allows for selective energizing of any of the tip 104, or either blade 332, 334, or both blades 332, 334. In some embodiments, a second electrical connection between the tip 104 and the energy source allows for selective energizing of the tip 104 separate from the first blade 332 or from either or both blades 332, 334. In some embodiments, a second electrical connection between the first blade 332 and the energy source allows for selective energizing of the second blade 334 separate from the first blade 332 or from the tip 104. In some embodiments, the cauterizer comprises a laser coagulation source, devices and energy sources for ultrasonic coagulation, devices and energy sources for high frequency coagulation, or devices and energy sources for high temperature plasma coagulation.

### Methods of Use

FIG. 5 shows an exemplary embodiment of the use of a cutting device in an ophthalmic procedure (e.g., an eye procedure, such as cataract surgery). In cutting a circular or a semi-circular hole in eye tissue, cutting head 100 is placed in the eye tissue with first sharp edge 334 placed against a surface to be cut. Thus, at a single time, a substantial portion (e.g., more than 25% or even more than 35%) of the length of the lens capsule which needs to be cut, e.g., more than 1-2 mm, is in contact with the sharp edge. Oscillator 108 is then operated until a cut in the size and shape of sharp edge 334 is achieved. Cutting head 100 is then turned over, such that second sharp edge 332 is placed against tissue to be cut. Oscillator 108 is then re-operated, so that a cut of the size and shape of sharp edge 334 is achieved. In some embodiments, the device cuts as the cutting head is rotated about its longitudinal axis, or as it is turned or moved. In such embodiments, the oscillator 108 remains active during such rotation, movement and/or turning.

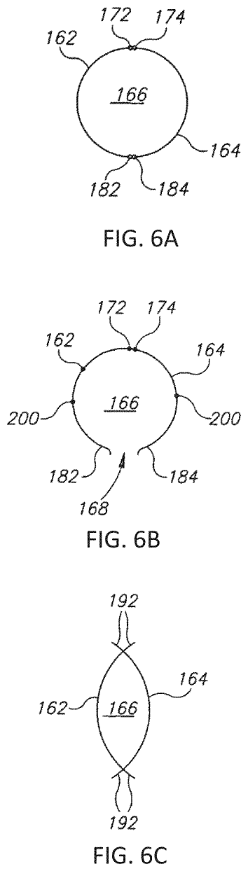

FIGS. 6A-6C are schematic illustrations of openings in the anterior chamber achieved using a cutting device, as disclosed herein, in accordance with exemplary embodiments of the invention. As shown in FIG. 6A, first and second cuts 162 and 164, achieved by sharp edges 332 and 334, respectively, define an incision 166. A top end 172 (in FIG. 6A) and a bottom end 182 of cut 162 substantially coincide with top and bottom ends 174 and 184 of cut 164, such that incision 166 is completely cut out of the underlying tissue and cuts 162 and 164 do not substantially extend into tissue not included in incision 166.

In FIG. 6B, the top ends 172 and 174 of cuts 162 and 164 substantially coincide, while the bottom ends 182 and 184 leave an uncut gap 168 between them. Optionally, the uncut gap 168 is not cut out in the cutting procedure, but rather is allowed to remain for the healing process. Alternatively, the uncut gap 168 is removed at a later stage using a third application of cutting head 100 or using any other cutting tool. In FIG. 6B, the incision 166 is larger than in FIG. 6A, due to gap 168.

In FIG. 6C, cuts 162 and 164 intersect, such that the cuts include external portions 192 that extend beyond that required in order to cut out incision 166. Generally, external portions 192 heal naturally and do not cause complications in the eye. In FIG. 6C, incision 166 is smaller than in FIG. 3A, due to the intersection of the cuts 162 and 164.

By selecting a relative layout of cuts 162 and 164, a physician determines a desired size and shape of incision 166. Thus, a single cutting device can be used to form incisions 166 of a plurality of different sizes. Referring to a farthest point 200 from the longitudinal axis of the cutting device, the distance between point 200 in cuts 162 and 164 defines, in some embodiments of the invention, the size and shape of incision 166. Optionally, markings 187 along cutting head 100 aid the physician in positioning the second sharp edge (e.g., 332) relative to the first cut (e.g., 164) in order to achieve a cut of a desired size. Alternatively, or additionally, cutting head 100 includes visible markings 180, which identify the end points of sharp edges 332 and 334.

In some embodiments, the method of use includes activating a cauterization feature of the device, which energizes the blade (either or both blade), tip, or any combination thereof, to cauterize tissue cut by such blade(s) or tip, simultaneously with the oscillation and/or vibration of the blade, or thereafter. The cauterization feature of the device may also be used simultaneously with the rotation of the head of the device as it rotates about the device axis, with or without oscillation simultaneously engaged.

The device, systems, and methods described herein are described in connection to an ophthalmic use, however, such devices and systems may also be used in other areas of any living animal, such as in removing tissue in vascular areas such as tonsillectomies, prostate surgery, urethral stricture repairs, ovary removals, breast surgery, various cosmetic surgeries, small tissue biopsies in many organs, etc. The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

When used in the following claims, the terms “comprise”, “include”, “have” and their conjugates mean “including but not limited to.” Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for cutting tissue of a subject, the device comprising:
a cutting head comprising
a distal tip comprising a first distal tip edge,
a proximal end, and
a first portion between the distal tip and the proximal end, the first portion comprising first edge and a second edge, at least one of which is a sharp edge, wherein the first distal tip edge of the distal tip forms a first angle with the first edge of the first portion of between 0 degrees and 180 degrees; and
an oscillator operatively coupled to the proximal end of the cutting head.

2. The device of claim 1, wherein the distal tip comprises a second distal tip edge and the first distal tip edge of the distal tip forms a second angle with a second distal tip edge of the distal tip of from 90 degrees to 180 degrees, between 90 degrees and 135 degrees, or from 135 degrees to less than 180.

3. The device of claim 1, wherein the first angle is between 0 degrees and 90 degrees, from 1 degree to 30 degrees, from 30 degrees to 45 degrees, from 45 degrees to 60 degrees, from 30 degrees to 60 degrees, or from 60 degrees to less than 90 degrees.

4. The device of any one of claims 1-3, wherein the first portion is curved.

5. The device of claim 4, wherein the first portion has a radius of curvature of at least 1 mm, at least 2 mm, at least 3 mm, at least 4 mm, at least 5 mm, at least 6 mm, at least 7 mm, at least 8 mm at least 9 mm, at least 10 mm, more than 10 mm, from 1 mm to 10 mm, from 2 mm to 9 mm, from 2 mm to 8 mm, from 2 mm to 7 mm, from 2 mm to 6 mm, from 2 mm to 5 mm, from 2 mm to 4 mm, or from 2 mm to 3 mm.

6. The device of claim 4, the first portion has a radius of curvature of 2.75 mm.

7. The device of any one of claims 4-6, wherein the first portion is semicircular.

8. The device of claim 1, the first edge of the first portion and the second edge of the first portion are both sharp edges.

9. The device of any one of claims 1-8, wherein the first distal tip edge of the distal tip is sharp.

10. The device of any one of claims 2-9, wherein the second distal tip edge of the distal tip is sharp.

11. The device of any one of claims 1-10, wherein a medial surface of the distal tip forms a second angle with a lateral surface of the distal tip of between 0 degrees and 90 degrees, from 1 degrees to 75 degrees, from 15 degrees to 85 degrees, from 20 degrees to 70 degrees, from 10 degrees to 45 degrees, from 30 degrees to 60 degrees, from 25 degrees to 65 degrees, from 15 degrees to 90 degrees, or from 15 degrees to 75 degrees.

12. The device of claim 11, wherein the second angle is determined using the medial surface and lateral surface along the distal tip length.

13. The device of any of any one of claims 1-12, wherein a third angle between the first distal tip edge and the second distal tip edge of the distal tip is less than 90 degrees.

14. The device of any one of claims 1-13, wherein the first portion comprises a medial surface connected to a lateral surface via at least two connecting surfaces, wherein at least one connecting surface of the device is a beveled surface.

15. The device of claim 14, wherein the two connecting surfaces of the device are beveled surfaces.

16. A device for cutting tissue of a subject, the device comprising:
a cutting head comprising a distal tip, a proximal end, and a first portion comprising a first sharp edge, the first portion being between the distal tip and the proximal end; and
an oscillator operatively coupled to the proximal end of the cutting head, wherein the cutting head has a medial surface, a lateral surface, a first connecting surface coupling the medial surface to the lateral surface, and a second connecting surface coupling the medial surface to the lateral surface.

17. The device of claim 16 wherein the first sharp edge is at the intersection of the medial surface and the first connecting surface, at the intersection of the medial surface and the second connecting surface, at the intersection of the lateral surface and the first connecting surface, at the intersection of the lateral surface and the second connecting surface, is part of the first connecting surface, or is part of the second connecting surface.

18. The device of claim 17, wherein the first sharp edge is substantially aligned with the medial surface of the cutting head or with the lateral surface of the cutting head.

19. The device of claim 17, wherein the first sharp edge is not substantially aligned with the medial surface of the cutting head or with the lateral surface of the cutting head.

20. The device of claim 18 or 19, wherein substantially aligned means that a distance from the medial surface or lateral surface to the first sharp edge is less than 2%, less than 5%, less than 10%, or less than 20% of a thickness of the cutting head measured perpendicularly in the transverse cross-section from the surface.

21. The device of any one of claims 16-20, wherein the first connecting surface is a beveled surface.

22. The device of any one of claims 16-21, wherein the second connecting surface is a beveled surface.

23. The device of any one of claims 16-22, wherein the first portion comprises a second sharp edge.

24. The device of claim 23, wherein the second sharp edge is at the intersection of the medial surface and the first connecting surface, at the intersection of the medial surface and the second connecting surface, at the intersection of the lateral surface and the first connecting surface, at the intersection of the lateral surface and the second connecting surface, is part of the first connecting surface, or is part of the second connecting surface.

25. The device of any one of claims 16-24, wherein the first portion of the cutting head comprises a trapezoidal transverse cross-section.

26. The device of claim 25, wherein the beveled surface forms an angle of from 0 degrees to 30 degrees, from 30 degrees to 45 degrees, from 0 degrees to 45 degrees, from 45 degrees to 90 degrees, from 45 degrees to 60 degrees, from 30 degrees to 60 degrees with the medial surface of the cutting head.

27. The device of claim 26, wherein the angle is of from 0 degrees to 30 degrees, from 30 degrees to 45 degrees, from 0 degrees to 45 degrees, from 45 degrees to 90 degrees, from 45 degrees to 60 degrees, from 30 degrees to 60 degrees.

28. The device of claim 16, wherein the first sharp edge is at the intersection of the medial surface and the beveled surface.

29. The device of claim 28, wherein the connecting surface forming the first sharp edge is a beveled surface, and wherein first sharp edge is formed by an angle between the medial surface and the beveled surface of between 0 degrees and 90 degrees, from 90 degrees to less than 180 degrees, from 90 degrees to 135 degrees, or from 135 degrees to less than 180 degrees with the lateral surface of the cutting head.

30. The device of claim 29, wherein the beveled surface forms an angle of from 0 degrees to 30 degrees, from 30 degrees to 45 degrees, from 0 degrees to 45 degrees, from 45 degrees to 90 degrees, from 45 degrees to 60 degrees, from 30 degrees to 60 degrees with the lateral surface of the cutting head.

31. The device of claim 30, wherein the first sharp edge is at the intersection of the lateral surface and the beveled surface.

32. The device of any one of claims 16-31, wherein the first portion is curved.

33. The device of claim 32, wherein the first portion has a radius of curvature of at least 1 mm, at least 2 mm, at least 3 mm, at least 4 mm, at least 5 mm, at least 6 mm, at least 7 mm, at least 8 mm at least 9 mm, at least 10 mm, more than 10 mm, from 1 mm to 10 mm, from 2 mm to 9 mm, from 2 mm to 8 mm, from 2 mm to 7 mm, from 2 mm to 6 mm, from 2 mm to 5 mm, from 2 mm to 4 mm, or from 2 mm to 3 mm.

34. The device of claim 32 or claim 33, wherein the curved portion comprises a radius of curvature of 2.75 mm.

35. The device of any one of claims 32-34, wherein the first portion is semicircular.

36. The device of any one of claims 16-35, wherein the distal tip is needle-shaped.

37. A device for cutting tissue of a subject, the device comprising:
a cutting head comprising a distal tip, a first portion, and a proximal end, the first portion comprising a first sharp edge;
an oscillator operatively coupled to the proximal end of the cutting head; and
a plurality of actuator controls, each actuator control configured to operate the oscillator.

38. The device of claim 37, wherein a first actuator control of the plurality of actuator controls is positioned on a housing of the device from 45 degrees to 180 degrees apart from a second actuator control of the plurality of actuator controls, measured in a circumferential arc about a longitudinal axis of the device.

39. The device of claim 38, wherein the first actuator control of the plurality of actuator controls is positioned on the housing about 90 degrees apart from the second actuator control, measured in a circumferential arc about the longitudinal axis of the device.

40. The device of claim 38, wherein the first actuator control of the plurality of actuators is positioned on the housing about 180 degrees apart from the second actuator control, measured in a circumferential arc about the longitudinal axis of the device.

41. The device of claim 38, wherein the first actuator control of the plurality of actuator controls is positioned on the housing about 90 degrees apart from the medial surface of a handle of the cutting head, measured in a circumferential arc about the longitudinal axis of the device.

42. The device of claim 37, wherein the oscillator moves the first sharp edge back and forth in a direction substantially parallel to a medial surface or a lateral surface of a handle of the cutting head, thereby moving the first sharp edge into tissue and away from tissue during use.

43. The device of claim 37, wherein the oscillator moves the first sharp edge back and forth in a direction substantially along a line intersecting the transverse and coronal planes of the device, thereby moving the first sharp edge into a subject and away from a subject during use.

44. The device of claim 37, wherein the oscillator moves the first sharp edge back and forth in a direction substantially along the longitudinal axis of the device.

45. The device of any one of claims 42-44, wherein the oscillator minimizes the perpendicular movement of the first sharp edge back and forth in a direction of a line intersecting the transverse and sagittal planes of the device such that the perpendicular movement of the first sharp edge is about 50% or less, less than 40% of, or less than 20% of as compared to the movement distance in the direction substantially parallel to the line intersecting the transverse and coronal planes of the device.

46. The device of claim 37, wherein the oscillator moves the first sharp edge at a rate of at least 300 Hz, at least 400 Hz, at least 500 Hz, at least 1000 Hz, at least 1500 Hz, at least 2000 Hz, at least 2500 Hz, at least 3000 Hz, at least 3500 Hz, at least 4000 Hz, at least 4500 Hz, or at least 5000 Hz.

47. The device of any one of claims 1-46, wherein the oscillator is configured to oscillate the cutting head at a rate of at least 100 Hz, at least 300 Hz, at least 1000 Hz, at least 3000 Hz, or at least 5000 Hz.

48. The device of any one of claims 1-47, further comprising a rotational actuation control configured to rotate the cutting head about a longitudinal axis of the device.

49. A device for cutting tissue of a subject, the device comprising:
a cutting head comprising a distal tip, a proximal end, a first portion comprising a first sharp edge positioned adjacent the distal tip, and a handle positioned proximal to the first portion and adjacent to the proximal end; and
a cutting device body having an oscillator housed therein and a handle connector configured to couple the oscillator to the handle of cutting head.

50. The device of claim 47, wherein the handle connector irreversibly couples the cutting head to the cutting device body.

51. The device of claim 49, wherein the handle connector comprises one or more of a chemical fixture, a mechanical fixture, or a friction joint

52. The device of claim 49, wherein the handle connector includes an opening with an inner dimension substantially sized to fit the handle thickness therein and includes a handle fixture flange extending from the wall at a distal end of the handle connector and into the opening of the connector.

53. The device of claim 52, wherein the cutting head comprises a handle fixture notch on the handle configured to align with the handle fixture flange in position and size when inserted in the handle connector opening.

54. The device of claim 52, wherein when inserted into the device, the handle deflects the fixture flange away from the longitudinal axis until the fixture flange reaches the notch, which prevents reverse movement of the handle out of the handle connector.

55. The device of any one of claims 42-54, wherein the handle comprises metal or stiff plastic having a Young's modulus greater than 3.0 GPa at room temperature and the handle connector comprises metal or stiff plastic having a Young's modulus greater than 3.0 GPa at room temperature, thereby generating a metal-to-metal interface, a metal-to-plastic interface, or a plastic-to plastic interface.

56. The device of claim 55, wherein the handle configured to outer dimensions are within 1%, 2%, 3%, 5%, 10%, <10%, <8%, <5%, <2%, <1%, <0.5%, <0.2%, 0.5% to 5%, 0.5% to 2%, 0.1% to 5%, or 0.1% to 10% of the inner dimensions of the handle connector.

57. The device of claim 53, wherein one or both of the handle connector and the handle comprise one or more alignment guides configured to ensure the handle inserts in the proper orientation relative to the cutting device body and the handle connector.

58. The device of claim 53, wherein one or both of the handle connector and the handle comprise one or more an insertion distance guides that provide visual or audible indication of proper insertion and fixation of the handle in the device body.

59. The device of any one of claims 1-58, wherein at least a portion of the cutting head has a thickness of 0.4 mm to 0.6 mm.

60. The device of any one of claims 1-59, wherein at least a portion of the cutting head has a thickness of 0.5 mm.

61. The device of claim 44, wherein the rotational actuation control comprises a lock.

62. The device of any one of claims 49-61, wherein the oscillator is configured to oscillate the cutting head at a rate of at least 100 Hz, at least 300 Hz, at least 1000 Hz, at least 3000 Hz, or at least 5000 Hz.

63. The device of any one of claims 1-62, comprising a cauterization energy source coupled to the cutting head configured to cauterize the tissue cut by the blade of the device by delivering cauterization energy to the first sharp edge of the cutting head.
